(19) United States
(12) Patent Application Publication
(10) Pub. No.: US 2024/0384352 A1
(43) Pub. Date: Nov. 21, 2024

(54) SPATIOTEMPORAL EVOLUTION OF TUMOR MICROENVIRONMENTS

(71) Applicant: ILLUMINA, INC., San Diego, CA (US)

(72) Inventors: Raakhee VIJAYARAGHAVAN, San Diego, CA (US); Chen ZHAO, San Diego, CA (US); Shile ZHANG, San Diego, CA (US); Li LIU, San Diego, CA (US); Mahdi GOLKARAM, San Diego, CA (US); Michael SALMANS, San Diego, CA (US)

(21) Appl. No.: 18/569,521

(22) PCT Filed: Mar. 6, 2023
(86) PCT No.: PCT/US2023/014587

§ 371 Date: Dec. 12, 2023

Related U.S. Application Data
(60) Provisional application No. 63/269,296, filed on Mar. 14, 2022.

(51) Int. Cl. C12Q 1/6886

(52) U.S. Cl. CPC C12Q 1/6886; C12Q 2600/106; C12Q 2600/112

(57) ABSTRACT

Described is a multi-regional, multi-omics approach to uncovering the mechanisms of immune escape in carcinoma. Immunotherapy with ant-PD1 and anti-CTLA4 elicits a heterogeneous immunological response across different regions of the tumor. Several genetic alterations associated with high intra-tumoral heterogeneity (ITH) shape tumor microenvironment (TME).

### PRIORITY AND CROSS-REFERENCE TO RELATED APPLICATIONS

This application claims the benefit of U.S. Provisional Application 63/269,296, filed on Mar. 14, 2022, which is hereby incorporated by reference in its entirety.

### BACKGROUND

### Field

Embodiments relates to systems and methods for determining intra-tumoral heterogeneity in a tumor microenvironment. More specifically, embodiments may relate to systems and methods of determining the efficacy of a immune checkpoint inhibitor on a tumor population.

### Description of the Related Art

Clear cell renal cell carcinoma (ccRCC) is the most common histological subtype of kidney cancer and has been described as one the most immunogenic tumor types (Liu et al., 2015). The efficacy of checkpoint inhibitor immunotherapy such as nivolumab, pembrolizumab and ipilimumab is well-established across cancer types with renal cell carcinoma among malignancies with one of the highest response rates to this immune checkpoint inhibitor (ICB) treatment (McDermott et al., 2018; Motzer et al., 2018). Yet, only a subset of these ccRCC patients has been found to derive a long-term clinical benefit. Several biomarkers have been suggested to determine the potential benefit of patients from ICB (Havel et al., 2019). Namely, the use of programmed cell death 1 (PD1) and its ligand (PD-L1) expression and tumor mutational burden (TMB) were approved by the United States Food and Drug Administration (FDA) for clinical use. Nevertheless, despite a low mutational load, ICB therapy has been found to induce durable benefits in ccRCC patients (Yarchoan et al., 2017).

### SUMMARY

In some embodiments, a method for determining an intra-tumoral heterogeneity (ITH) in a tumor microenvironment (TME) of a cancer is provided.

In some embodiments, the method for determining an intra-tumoral heterogeneity (ITH) in a tumor microenvironment (TME) of a cancer comprises providing a biopsy sample from a cancer patient, ascertaining a weighted genome instability index (wGII) based on a mutational frequency in a plurality of regions within the biopsy sample, comparing the wGII to a first median value and classifying the plurality of regions within the biopsy sample as having either higher wGII than the first median value or lower wGII than the first median value, and analyzing a plurality of parameters in the plurality of regions within the biopsy sample, classified as having either higher wGII than the first median value or lower wGII than the first median value, to determine an ITH index of each of the plurality of parameters of each of the plurality of regions within the biopsy sample.

In some embodiments, the method for determining an intra-tumoral heterogeneity (ITH) in a tumor microenvironment (TME) of a cancer further comprises comparing the ITH index of each of the plurality of parameters to a second median value, and classifying each of the plurality of parameters as having either a higher ITH index than the second median value or a lower ITH index than the second median value.

In some embodiments of the method for determining an intra-tumoral heterogeneity (ITH) in a tumor microenvironment (TME) of a cancer, a higher ITH index than the second median value correlates with a higher wGII in each of the plurality of regions within the biopsy sample.

In some embodiments of the method for determining an intra-tumoral heterogeneity (ITH) in a tumor microenvironment (TME) of a cancer, a lower ITH index than the second median value correlates with a lower wGII in each of the plurality of regions within the biopsy sample.

In some embodiments of the method for determining an intra-tumoral heterogeneity (ITH) in a tumor microenvironment (TME) of a cancer, the cancer is clear cell renal cell carcinoma (ccRCC).

In some embodiments of the method for determining an intra-tumoral heterogeneity (ITH) in a tumor microenvironment (TME) of a cancer, the biopsy sample is a nephrectomy sample.

In some embodiments of the method for determining an intra-tumoral heterogeneity (ITH) in a tumor microenvironment (TME) of a cancer, analyzing the plurality of parameters in the plurality of regions within the biopsy sample is performed by collecting at least one of DNA, RNA, cellular fractions, tissue sections, and tissue extracts for each of the plurality of regions within the biopsy sample.

In some embodiments of the method for determining an intra-tumoral heterogeneity (ITH) in a tumor microenvironment (TME) of a cancer, the plurality of parameters comprises genomic analysis, transcriptomic analysis, TCR analysis, immune cell analysis, metabolomics analysis, pathology analysis, myeloid signature analysis, JAVELIN signature analysis, effector T cell signature analysis, antigen presentation signature analysis, and angiogenic signature analysis.

In some embodiments, the method for determining an intra-tumoral heterogeneity (ITH) in a tumor microenvironment (TME) of a cancer further comprises correlating the ITH index of each of the plurality of parameters with at least one ccRCC evolutionary subtype.

In some embodiments of the method for determining an intra-tumoral heterogeneity (ITH) in a tumor microenvironment (TME) of a cancer, the at least one ccRCC evolutionary subtype comprises a VHL wildtype, a VHL monodriver, multiple clonal driver, a BAP1 driver, or PBRM1 driven tumors.

In some embodiments of the method for determining an intra-tumoral heterogeneity (ITH) in a tumor microenvironment (TME) of a cancer, the PBRM1 driven tumors comprise PBRM1→SETD2, PBRM1→SCNA, and PBRM1→PT3K.

In some embodiments of the method for determining an intra-tumoral heterogeneity (ITH) in a tumor microenvironment (TME) of a cancer, the VHL monodriver and multiple clonal driver subtypes correlate with an ITH index that is lower than the second median value.

In some embodiments of the method for determining an intra-tumoral heterogeneity (ITH) in a tumor microenvironment (TME) of a cancer, PBRM1 driven tumors, SETD2 mutations, loss of heterozygosity (LOH) in Human Leukocyte Antigen (HLA), and loss of CDKN2A/B copy number correlates with an ITH index that is higher than the second median value.

In some embodiments of the method for determining an intra-tumoral heterogeneity (ITH) in a tumor microenvironment (TME) of a cancer, PBRM1 driven tumors are associated with elevated HERV expression.

In some embodiments, the method for determining an intra-tumoral heterogeneity (ITH) in a tumor microenvironment (TME) of a cancer further comprises ascertaining neoantigen heterogeneity by counting 8-11 amino acids length neoantigens in the plurality of regions within the biopsy sample.

In some embodiments of the method for determining an intra-tumoral heterogeneity (ITH) in a tumor microenvironment (TME) of a cancer, an ITH index that is higher than the second median value is associated with higher neoantigen editing in the plurality of regions within the biopsy sample.

In some embodiments of the method for determining an intra-tumoral heterogeneity (ITH) in a tumor microenvironment (TME) of a cancer, an ITH index that is lower than the second median value is associated with lower neoantigen editing in the plurality of regions within the biopsy sample.

In some embodiments, the method for determining an intra-tumoral heterogeneity (ITH) in a tumor microenvironment (TME) of a cancer further comprises administering ICB treatment to the at least one patient and ascertaining neoantigen heterogeneity in the plurality of regions within the biopsy sample prior to and after administering the ICB treatment.

In some embodiments of the method for determining an intra-tumoral heterogeneity (ITH) in a tumor microenvironment (TME) of a cancer, ascertaining neoantigen heterogeneity comprises counting 8-11 amino acids length neoantigens prior to and after administering the ICB treatment.

In some embodiments of the method for determining an intra-tumoral heterogeneity (ITH) in a tumor microenvironment (TME) of a cancer, a deletion of neoantigens after administering the ICB treatment is indicative of neoantigen editing by the ICB treatment.

In some embodiments of the method for determining an intra-tumoral heterogeneity (ITH) in a tumor microenvironment (TME) of a cancer, a deletion of neoantigens after administering the ICB treatment is indicative of selective pressure against hydrophobic residues and selection in favor of hydrophilic residues.

In some embodiments of the method for determining an intra-tumoral heterogeneity (ITH) in a tumor microenvironment (TME) of a cancer, a deletion of neoantigens after administering the ICB treatment is indicative of selective pressure against Phenylalanine and selection in favor of Arginine and Glutamic acid.

In some embodiments of the method for determining an intra-tumoral heterogeneity (ITH) in a tumor microenvironment (TME) of a cancer, a deletion of neoantigens after administering the ICB treatment correlates with an ITH index that is higher than the second median value.

In some embodiments of the method for determining an intra-tumoral heterogeneity (ITH) in a tumor microenvironment (TME) of a cancer, a deletion of neoantigens after administering the ICB treatment correlates with an immunosuppressive TME.

In some embodiments of the method for determining an intra-tumoral heterogeneity (ITH) in a tumor microenvironment (TME) of a cancer, the ICB treatment is selected from the group consisting of Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

In some embodiments of the method for determining an intra-tumoral heterogeneity (ITH) in a tumor microenvironment (TME) of a cancer, an ITH index that is higher than the second median value correlates with high myeloid signature and low effector T cell signature.

In some embodiments of the method for determining an intra-tumoral heterogeneity (ITH) in a tumor microenvironment (TME) of a cancer, an ITH index that is higher than the second median value correlates with low antigen presentation signature.

In some embodiments of the method for determining an intra-tumoral heterogeneity (ITH) in a tumor microenvironment (TME) of a cancer, an ITH index that is higher than the second median value correlates with reduced TCR diversity.

In some embodiments of the method for determining an intra-tumoral heterogeneity (ITH) in a tumor microenvironment (TME) of a cancer, an ITH index that is lower than the second median value correlates with the biopsy being sparsely infiltrated by tumor infiltrating lymphocytes (TILs) and/or being infiltrated by stromal TILs.

In some embodiments of the method for determining an intra-tumoral heterogeneity (ITH) in a tumor microenvironment (TME) of a cancer, an ITH index that is higher than the second median value correlates with the biopsy being infiltrated by substantial levels of both epithelial and stromal TILs.

In some embodiments of the method for determining an intra-tumoral heterogeneity (ITH) in a tumor microenvironment (TME) of a cancer, infiltration of the biopsy by substantial levels of both epithelial and stromal TILs is correlated with an immune evasion/escape gene signature.

In some embodiments of the method for determining an intra-tumoral heterogeneity (ITH) in a tumor microenvironment (TME) of a cancer, immune evasion/escape is correlated with HLA LOH and CDKN2A/B loss in the cellular fractions collected from the plurality of regions within the biopsy sample.

In some embodiments of the method for determining an intra-tumoral heterogeneity (ITH) in a tumor microenvironment (TME) of a cancer, immune evasion/escape is correlated with HLA LOH and CDKN2A/B loss in a cellular fraction collected from peripheral blood of the patient.

In some embodiments of the method for determining an intra-tumoral heterogeneity (ITH) in a tumor microenvironment (TME) of a cancer, the genomic analysis comprises small variant calling, evaluation of somatic copy number alterations, allele specific copy number calling, HLA typing, and in silico binding prediction of putative neoantigens.

In some embodiments of the method for determining an intra-tumoral heterogeneity (ITH) in a tumor microenvironment (TME) of a cancer, the transcriptomic analysis comprises quantification of gene expression data, a gene expression microarray, RT-PCR, and RNA-Seq.

In some embodiments of the method for determining an intra-tumoral heterogeneity (ITH) in a tumor microenvironment (TME) of a cancer, the TCR analysis comprises T cell clonotyping, T cell diversity estimation using a diversity index such as Shannon Entropy index, Simpson's Diversity index, and Berger Parker index.

In some embodiments of the method for determining an intra-tumoral heterogeneity (ITH) in a tumor microenvironment (TME) of a cancer, the immune cell analysis comprises gene signature analysis, such as, for example, a tumor microenvironment gene signature analysis. In some embodiments, the gene signature analysis comprises use of a gene set enrichment analysis, for example a single sample Gene Set Enrichment Analysis (ssGSEA) method, as is known in the art and exemplified by Barbie D A, Tamayo P, et al. Systematic RNA interference reveals that oncogenic KRAS-driven cancers require TBK1.

In some embodiments of the method for determining an intra-tumoral heterogeneity (ITH) in a tumor microenvironment (TME) of a cancer, the metabolomics analysis comprises quantification of major metabolites in a tumor tissue sample using liquid chromatography-mass spectrometry (LC-MS) and tandem mass spectroscopy (MS/MS) for quantification of metabolites in the tumor tissue sample.

In some embodiments of the method for determining an intra-tumoral heterogeneity (ITH) in a tumor microenvironment (TME) of a cancer, the pathology analysis comprises tumor-stroma-immune grading using a pathology method to classify tumors to N-TIL (tumors sparsely infiltrated by TILs), S-TIL (tumors dominated by stromal TILs), and ES-TIL (tumors with substantial levels of both epithelial and stromal TILs).

In some embodiments of the method for determining an intra-tumoral heterogeneity (ITH) in a tumor microenvironment (TME) of a cancer, the myeloid signature analysis comprises ssGSEA analysis to evaluate enrichment of one or more of the following genes: IL6, CXCL1, CXCL2, CXCL3, CXCL8, and PTGS2.

In some embodiments of the method for determining an intra-tumoral heterogeneity (ITH) in a tumor microenvironment (TME) of a cancer, the JAVELIN signature analysis comprises ssGSEA analysis to evaluate enrichment of one or more of the following genes: NRARP, NRXN3, CALCRL, TEK, ECSCR, PTPRB, CD34, RAMP2, KDR, NOTCH4, FLT1, GJA5, TBX2, HEY2, ARHGEF15, SMAD6, AQP1, GATA2, ENPP2, ATP1A2, EDNRB, VIP, KCNAB1, RAMP3, CACNB2, and CASQ2.

In some embodiments of the method for determining an intra-tumoral heterogeneity (ITH) in a tumor microenvironment (TME) of a cancer, the effector T cell signature analysis comprises ssGSEA analysis to evaluate enrichment of one or more of the following genes: CD8A, EOMES, PRF1, IFNG, and CD274.

In some embodiments of the method for determining an intra-tumoral heterogeneity (ITH) in a tumor microenvironment (TME) of a cancer, the antigen presentation signature analysis comprises ssGSEA analysis to evaluate enrichment of one or more of the following genes: HLA-A, HLA-B, HLA-C, B2M, TAP1, TAP2, and TAPBP.

In some embodiments of the method for determining an intra-tumoral heterogeneity (ITH) in a tumor microenvironment (TME) of a cancer, the angiogenic signature analysis comprises ssGSEA analysis to evaluate enrichment of one or more of the following genes: VEGFA, KDR, ESM1, PECAM1, ANGPTL4, and CD34.

In some embodiments of the method for determining an intra-tumoral heterogeneity (ITH) in a tumor microenvironment (TME) of a cancer, the immune evasion/escape gene signature comprises ssGSEA analysis to evaluate enrichment of one or more of the following genes: TIMP1, PXDN, COL15A1, OLFML2B, COL5A2, DLX5, SOXI1, KLHDC8A, UNC5A, ADAMTS14, MMP11, and FN1.

In some embodiments, a method of predicting an outcome of immune checkpoint inhibitor (ICB) therapy is provided.

In some embodiments, the method of predicting an outcome of immune checkpoint inhibitor (ICB) therapy comprises providing a biopsy sample from a cancer patient, ascertaining a weighted genome instability index (wGII) based on a mutational frequency in a plurality of regions within the biopsy sample, comparing the wGII to a first median value and classifying the plurality of regions within the biopsy sample as having either a higher wGII than the first median value or a lower wGII than first median value, analyzing a plurality of parameters in the plurality of regions within the biopsy sample, classified as having either higher wGII than median or lower wGII than median, to determine an ITH index of each of the plurality of parameters of each of the plurality of regions within the biopsy sample, comparing the ITH index of each of the plurality of parameters to a second median value, and classifying each of the plurality of parameters as having either higher ITH index than the second median value or a lower ITH index than the second median value.

In some embodiments of the method of predicting an outcome of immune checkpoint inhibitor (ICB) therapy, a higher ITH index than the second median value is predictive of the patient being nonresponsive to ICB therapy, and

In some embodiments of the method of predicting an outcome of immune checkpoint inhibitor (ICB) therapy, lower ITH index than the second median value is predictive of the patient being responsive to ICB therapy.

In some embodiments of the method of predicting an outcome of immune checkpoint inhibitor (ICB) therapy, the cancer is clear cell renal cell carcinoma (ccRCC).

In some embodiments of the method of predicting an outcome of immune checkpoint inhibitor (ICB) therapy, the biopsy sample is a nephrectomy sample.

In some embodiments of the method of predicting an outcome of immune checkpoint inhibitor (ICB) therapy, analyzing the plurality of parameters in the plurality of regions within the biopsy sample is performed by at least one of collecting DNA, RNA, cellular fraction, tissue section, and tissue extraction for each of the plurality of regions within the biopsy sample.

In some embodiments of the method of predicting an outcome of immune checkpoint inhibitor (ICB) therapy, the ICB treatment is selected from the group consisting of: Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

In some embodiments, the method of predicting an outcome of immune checkpoint inhibitor (ICB) therapy and/or method for determining an intra-tumoral heterogeneity (ITH) in a tumor microenvironment (TME) of a cancer further comprises administering ICB treatment to the cancer patient if the ITH index is lower than the second median value.

In some embodiments, the method of predicting an outcome of immune checkpoint inhibitor (ICB) therapy and/or method for determining an intra-tumoral heterogeneity (ITH) in a tumor microenvironment (TME) of a cancer further comprises administering a non-ICB cancer therapy and not administering an ICB treatment to the cancer patient if the ITH index is higher than the second median value.

### BRIEF DESCRIPTION OF THE DRAWINGS

FIG. 1A shows the landscape of immune escape intra-tumoral heterogeneity in clear cell renal cell carcinoma.

FIG. 1B is a diagram illustrating the layout of an experiment and resultant data summary from one embodiment of the invention showing some patient characteristics and the study design.

FIGS. 2A-2D show a series of charts detailing the landscape of ITH in ccRCC.

FIG. 2A shows mutational event per sample for all regions of 31 patients.

FIG. 2B shows a comparison between mutational frequency observed in this cohort and TRACERx Renal.

FIG. 2C shows a heatmap shows ITH high vs low classification across data type. Annotation illustrates somatic alterations, evolutionary subtypes, and treatment status of patients. A patient is annotated as wildtype if all regions are wild type for that alteration.

FIG. 2D shows an association between PBRM1 and ITH.

FIGS. 3A-3L show a series of chart showing the landscape of heterogeneity of neoantigen editing.

FIG. 3A shows change in neoantigen counts (compared to pre-treatment) but not TMB illustrates selective pressure and immuno-editing.

FIGS. 3B and 3C show one sample Wilcox test P (compared to zero) of immuno-editing in an HLA intact patient through reduced neoantigen expression.

FIG. 3D shows clonality of neoantigen depletion.

FIG. 3E shows immuno-editing with amino acid resolution. Higher Phenylalanine (F) depletion compared to Glutamic Acid (E) and Arginine (R) suggests immune selection.

FIGS. 3F and 3G show association between the fraction of neoantigens edited, ITH, and immune signatures. In FIG. 3G, correlations are calculated across different regions of the same patient for all patients with >3 RNA samples were available.

FIG. 3H shows association between antigen presentation machinery (APM), effector T cell (Teff) and myeloid gene signatures and ITH.

FIG. 3I shows that PBRM1 mutation is associated with elevated mutational count upon treatment.

FIGS. 3J and 3K show that HERVs are enriched in tumors compared to normal samples and are associated with treatment.

FIG. 3L shows HERV depletion association with myeloid signature.

FIGS. 4A-4D show the branch evolution demonstrating immune evasion.

FIG. 4A shows an evolutionary tree illustrates tumors can exploit concurrent HLA LOH and CDKN2A/B loss to escape immune surveillance.

FIG. 4B shows co-occurrence of HLA LOH and CDKN2A/B can be seen both across regions and patients.

FIG. 4C shows that the fraction of neoantigens edited is strongly associated with reduced TCR diversity.

FIG. 4D shows regions of tumors associated with immune escape depict a distinct pathology where colocalization of TILs and stroma can be observed. These regions demonstrate an elevated immune evasion gene signature. RA, RB, RC, RD, and RE denote different regions of a tumor sample.

FIG. 5A shows circos plot illustrating the fraction of shared T cell clonotypes between tissue and different time points on therapy.

FIG. 5B shows clonotype tracking shows clonal expansion and contraction in patient NIVO20.

FIG. 5C shows dynamic of PBMC TCR diversity on therapy (different lines represent different patients).

FIG. 5D shows an association between tissue and PBMC T cell clonotype overlap and ITH before and on therapy.

FIG. 5E shows an association between PBMC TCR diversity and richness and ITH.

FIGS. 5F-5J show association between PBMC TCR diversity and germline and somatic features. In FIG. 5J, PBRM1→SETD2 Wilcox test compares TCRs of this subtype with others. Likewise, PBRM1→PI3K Wilcox test compares TCR of this subtype with others except PBRM1→SETD2. P value is corrected for multiple comparisons.

FIG. 6A shows WGCNA was used to extract modules describing inflammation (JAVELIN), angiogenesis, and immune escape.

FIG. 6B shows modules “black”, “salmon”, and “magenta” are associated with previously described signatures, JAVELIN, angiogenesis and myeloid/stroma.

FIG. 6C shows immune escape signature is strongly associated with PTEN alteration in 2 independent cohorts.

FIG. 6D shows that scRNAseq demonstrates an enrichment of escape signature in stroma and myeloid cells. (left) Single cells are annotated by cell type and (right) FN1 and TIMIP (2 out of 12 genes in escape signature with expression above scRNAseq limit of detection) are highlighted to illustrate the cell type enrichment of this signature.

FIG. 7A shows an association between ITH and TME. Pie charts are organized to roughly reflect the location from where each biopsy is collected. The size of each pie chart represents tumor size (1—tumor regression) and pieces of each pie chart corresponds to the average ES/S/N observed for each region across all ITH high vs ITH low patients.

FIG. 7B shows an association between ITH and tumor regression.

FIG. 7C shows immune escape signature correlates with colocalization of immune infiltrates into stroma and epithelium.

FIG. 7D shows survival analysis shows the association between gene signatures obtained in this study and clinical outcome of different independent retrospective trials.

FIG. 8 shows boxplots of correlation between RNA ITH, metabolome ITH and T cell diversity.

FIG. 9 shows ccRCC evolutionary subtypes and their association with ITH.

FIG. 10 shows association between inflammation, HLA LOH and ITH.

FIG. 11A shows boxplots show total and change compared to pre-treatment (when sample was available) for mutational count, and neoantigen count across different regions of all patients.

FIG. 11B shows a boxplot of ITH of HERVs across different regions of all patients.

FIG. 12A shows boxplots of association between neoantigen depletion and ccRCC driver mutations.

FIG. 12B shows a boxplot of a comparison between SNPs neoantigen editing and INDEL depletion.

FIG. 13A shows an association between HERV expression and immune signatures.

FIG. 13B is a boxplot showing that PBRM1 mutations are associated with elevated HERV expression.

FIG. 13C shows a boxplot of an association between ClearCode34 classes and HERV expression.

FIG. 13D shows a boxplot of an association between ClearCode34 classes and neoantigen editing.

FIG. 14 shows hierarchical clustering of TCR clonotypes across different regions of patients where tissue TCRseq data was available.

FIG. 15 are boxplots showing association between immune escape signature, treatment, and ITH.

FIGS. 16A and 16B show boxplots of validation of escape signature in independent cohorts (IMmotion151). FIG. 16A shows box plots of escape signature is associated with improved survival in patients treated with ICB but not sunitinib. FIG. 16B shows a boxplot of escape signature is associated with CDKN2A/B alteration in Immotion151.

FIG. 17 shows boxplots of the relationship between an escape gene signature and treatment outcome in different clinical trials. HRs are calculated for each threshold for ICB or ICB in combination with TKI arms in JAVELIN Renal 101, IMmotion151, and CheckMate 009, 010, 025.

### DETAILED DESCRIPTION

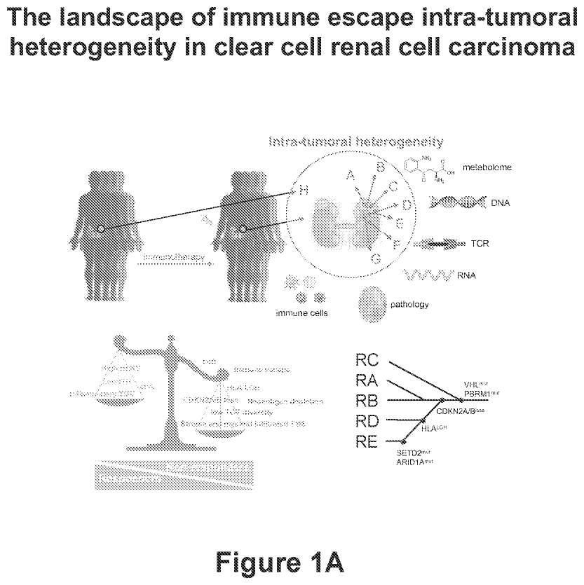

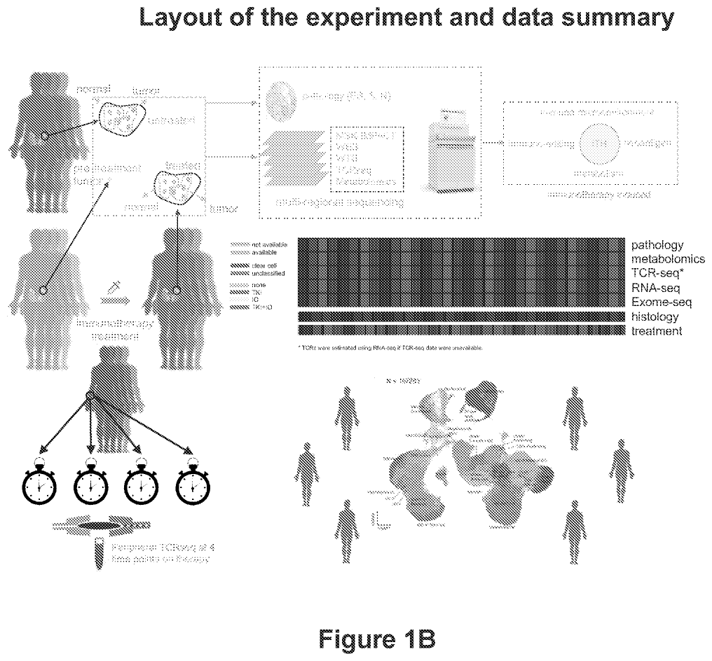

Previous studies have highlighted the molecular determinants of response to immune checkpoint inhibitors (ICBs). Embodiments relate to systems and methods of utilizing multi-regional multi-omics to uncover the mechanisms of immune escape in clear cell renal cell carcinoma and other cancers and predict the outcome of ICB therapy on tumor patients. According to some embodiments, a-PD1 and a-CTLA4 immunotherapy elicits a heterogeneous immunological response across different regions of the tumor. Moreover, intra-tumoral heterogeneity (ITH) correlates across genomic, transcriptomic, metabolomic, immune and TME landscapes which can contribute to clinical outcome of ICB immunotherapy (FIG. 1A).

Some embodiments relate to the discovery of several genetic alterations associated with high ITH and reveal how these genetic alterations shape the tumor microenvironment (TME). Without limitations, mutations in PBRM1, CDKN2A/B somatic copy number loss, PTEN loss and HLA loss of heterozygosity (LOH) enable certain tumor subpopulations to escape immune surveillance through neoantigen depletion and myeloid activation resulting in reduced peripheral blood T cell receptor (TCR) diversity. Thus, embodiments relate to analyzing these genetic alterations to predict the outcome of ICB therapy on tumor patients. In some embodiments, a signature associated with immune escape is linked to stroma and epithelium infiltrated T lymphocytes which can render ICB therapy ineffective. This signature strongly predicts the response to ICB treatment in a tumor patient and the effectiveness of targeted therapies in several independent retrospective cohorts.

A multi-regional multi-omics approach was utilized to portray the evolution of neoantigen pruning. Some embodiments show how the crosstalk between tumor and TME determines immune escape and yield a heterogenous immunological response to ICB treatment. Neoantigen editing occurs as a consequence of external selective pressures imposed by the immune system and can result in loss of immunogenic neoantigens. Among several escape mechanisms, some embodiments reveal concurrent subclonal HLA LOH and CDKN2A/B loss, and HERV downregulation are associated with neoantigen depletion. In addition, gene signature associated with immune escape was strongly associated with PTEN loss which can lead to an immunosuppressive TME in independent cohorts. These distinct genomic alterations can lead to reduced T cell diversity and can be detected in PBMC. Moreover, tumor cells exploit neoantigen pruning to modulate TME through myeloid activation and stroma associated signaling which further facilitate immune evasion. This signature describes a distinct immunophenotype where extensive epithelial/stromal TILs colocalization can be observed as previously shown in ovarian cancer (Zhang et al., 2018). Therefore, TIL abundance alone is an insufficient predictor of immunological response, and immune evasion through neoantigen pruning can render ICB treatment ineffective. Some embodiments shed light on how immune escape can lead to elevate ITH at genomic, transcriptomic, metabolomic and several features of TME and negatively impacts response to ICB treatment. In some embodiments, the immune escape signature was validated in several independent cohorts and demonstrated that this signature correlated with durable benefit to ICB treatment alone or in combination with TKI therapy.

### INTRODUCTION

FIG. 1B shows the overview and layout of how experiments were performed for the present study. As described in more detail below, multiregional multi-omics was performed on 31 patients. Seven out of the 31 patients were untreated and the rest were treated with ICB or in combination with TKI. TCRseq of PBMC was performed at four time points on therapy for a subset of patients. Moreover, scRNAseq data for 6 out 31 patients were available from Krishna et al., 2021. In addition, pathological review was performed to assign N-TIL (tumors sparsely infiltrated by TILs), S-TIL (tumors dominated by stromal TILs), and ES-TIL (tumors with substantial levels of both epithelial and stromal TILs) classes to a subset of patients. The present study demonstrated that genetic alterations such as allele specific HLA loss (which co-occurs with CDKN2A/B loss) together with neoantigen depletion can lead to reduced T cell diversity and are key mechanisms of immune evasion. In some embodiments, immune escape is associated with ITH, myeloid activation and stroma enriched TME.

### Methods for Determining an Intra-Tumoral Heterogeneity (ITH) in a Tumor Microenvironment (TME) of a Cancer

In some embodiments, a method for determining an intra-tumoral heterogeneity (ITH) in a tumor microenvironment (TME) of a cancer is provided.

In some embodiments, the method comprises providing a biopsy sample from a cancer patient, ascertaining a weighted genome instability index (wGII) based on a mutational frequency in a plurality of regions within the biopsy sample, comparing the wGII to a first median value and classifying the plurality of regions within the biopsy sample as having either higher wGII than the first median value or lower wGII than the first median value, and analyzing a plurality of parameters in the plurality of regions within the biopsy sample, classified as having either higher wGII than the first median value or lower wGII than the first median value, to determine an ITH index of each of the plurality of parameters of each of the plurality of regions within the biopsy sample.

In some embodiments, the method further comprises comparing the ITH index of each of the plurality of parameters to a second median value, and classifying each of the plurality of parameters as having either a higher ITH index than the second median value or a lower ITH index than the second median value. A higher ITH index than the second median value correlates with a higher wGII in each of the plurality of regions within the biopsy sample. A lower ITH index than the second median value correlates with a lower wGII in each of the plurality of regions within the biopsy sample.

In some embodiments of the method, the cancer is clear cell renal cell carcinoma (ccRCC).

In some embodiments of the method, the biopsy sample is a nephrectomy sample.

In some embodiments, other cancers are also contemplated and within the scope of the disclosure. Non-limiting examples include selected from the group consisting of colorectal (CRC), breast adenocarcinoma, pancreatic adenocarcinoma, lung carcinoma, prostate cancer, glioblastoma multiform, hormone refractory prostate cancer, solid tumor malignancies such as colon carcinoma, non-small cell lung cancer (NSCLC), anaplastic astrocytoma, bladder carcinoma, sarcoma, ovarian carcinoma, rectal hemangiopericytoma, pancreatic carcinoma, advanced cancer, cancer of large bowel, stomach, pancreas, ovaries, melanoma, pancreatic cancer, colon cancer, bladder cancer, hematological malignancies, squamous cell carcinomas, breast cancer, glioblastoma, brain neoplasms, pilocytic astrocytoma, diffuse astrocytoma, anaplastic astrocytoma, brain stem gliomas, glioblastomas multiforme, meningioma, ependymomas, oligodendrogliomas, mixed gliomas, pituitary tumors, craniopharyngiomas, germ cell tumors, pineal region tumors, medulloblastomas, and primary CNS lymphomas.

In some embodiments of the method, the biopsy sample is a sample from colorectal (CRC), breast adenocarcinoma, pancreatic adenocarcinoma, lung carcinoma, prostate cancer, glioblastoma multiform, hormone refractory prostate cancer, solid tumor malignancies such as colon carcinoma, non-small cell lung cancer (NSCLC), anaplastic astrocytoma, bladder carcinoma, sarcoma, ovarian carcinoma, rectal hemangiopericytoma, pancreatic carcinoma, advanced cancer, cancer of large bowel, stomach, pancreas, ovaries, melanoma, pancreatic cancer, colon cancer, bladder cancer, hematological malignancies, squamous cell carcinomas, breast cancer, glioblastoma, brain neoplasms, pilocytic astrocytoma, diffuse astrocytoma, anaplastic astrocytoma, brain stem gliomas, glioblastomas multiforme, meningioma, ependymomas, oligodendrogliomas, mixed gliomas, pituitary tumors, craniopharyngiomas, germ cell tumors, pineal region tumors, medulloblastomas, and primary CNS lymphomas.

Non-limiting examples of anti-cancer chemotherapeutics include Cyclophosphamide, methotrexate, 5-fluorouracil, vinorelbine, Doxorubicin, cyclophosphamide, Docetaxel, doxorubicin, cyclophosphamide, Doxorubicin, bleomycin, vinblastine, dacarbazine, Mustine, vincristine, procarbazine, prednisolone, Cyclophosphamide, doxorubicin, vincristine, prednisolone, Bleomycin, etoposide, cisplatin, Epirubicin, cisplatin, 5-fluorouracil, Epirubicin, cisplatin, capecitabine, Methotrexate, vincristine, doxorubicin, cisplatin, Cyclophosphamide, doxorubicin, vincristine, vinorelbine, 5-fluorouracil, folinic acid, and oxaliplatin.

Non-limiting examples of checkpoint inhibitors include Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

In some embodiments of the method of predicting, the cancer is selected from the group consisting of colorectal (CRC), breast adenocarcinoma, pancreatic adenocarcinoma, lung carcinoma, prostate cancer, glioblastoma multiform, hormone refractory prostate cancer, solid tumor malignancies such as colon carcinoma, non-small cell lung cancer (NSCLC), anaplastic astrocytoma, bladder carcinoma, sarcoma, ovarian carcinoma, rectal hemangiopericytoma, pancreatic carcinoma, advanced cancer, cancer of large bowel, stomach, pancreas, ovaries, melanoma, pancreatic cancer, colon cancer, bladder cancer, hematological malignancies, squamous cell carcinomas, breast cancer, glioblastoma, brain neoplasms, pilocytic astrocytoma, diffuse astrocytoma, anaplastic astrocytoma, brain stem gliomas, glioblastomas multiforme, meningioma, ependymomas, oligodendrogliomas, mixed gliomas, pituitary tumors, craniopharyngiomas, germ cell tumors, pineal region tumors, medulloblastomas, and primary CNS lymphomas.

In some embodiments of the method of predicting, the anti-cancer treatment is selected from the group consisting of surgery, radiation therapy, chemotherapy, immunotherapy, targeted therapy, hormone therapy, stem cell transplant, cytokine therapy, gene therapy, cell therapy, phototherapy, thermotherapy, and sound therapy.

In some embodiments of the method of predicting, the anti-cancer treatment comprises an anti-cancer chemotherapeutic selected from the group consisting of Cyclophosphamide, methotrexate, 5-fluorouracil, vinorelbine, Doxorubicin, cyclophosphamide, Docetaxel, doxorubicin, cyclophosphamide, Doxorubicin, bleomycin, vinblastine, dacarbazine, Mustine, vincristine, procarbazine, prednisolone, Cyclophosphamide, doxorubicin, vincristine, prednisolone, Bleomycin, etoposide, cisplatin, Epirubicin, cisplatin, 5-fluorouracil, Epirubicin, cisplatin, capecitabine, Methotrexate, vincristine, doxorubicin, cisplatin, Cyclophosphamide, doxorubicin, vincristine, vinorelbine, 5-fluorouracil, folinic acid, and oxaliplatin.

In some embodiments of the method of predicting, the cell-type specific markers are selected from the group consisting of: human endogenous retroviral (HERV) gene expression markers, tumor infiltrating lymphocyte (TIL) markers, microsatellite instability (MSI) status markers, and tumor mutational burden (TMB) markers.

In some embodiments of the method of predicting, the cell-type specific markers comprise markers associated with one or more of CD8+ T, CD4+ T, and CD19+ B cells.

In some embodiments of the method, analyzing the plurality of parameters in the plurality of regions within the biopsy sample is performed by collecting at least one of DNA, RNA, cellular fractions, tissue sections, and tissue extracts for each of the plurality of regions within the biopsy sample.

In some embodiments of the method, the plurality of parameters comprises genomic analysis, transcriptomic analysis, TCR analysis, immune cell analysis, metabolomics analysis, pathology analysis, myeloid signature analysis, JAVELIN signature analysis, effector T cell signature analysis, antigen presentation signature analysis, and angiogenic signature analysis.

In some embodiments of the method, the plurality of parameters is selected from the group consisting of genomic analysis, transcriptomic analysis, TCR analysis, immune cell analysis, metabolomics analysis, pathology analysis, myeloid signature analysis, JAVELIN signature analysis, effector T cell signature analysis, antigen presentation signature analysis, and angiogenic signature analysis.

In some embodiments of the method, the plurality of parameters comprises two or more of genomic analysis, transcriptomic analysis, TCR analysis, immune cell analysis, metabolomics analysis, pathology analysis, myeloid signature analysis, JAVELIN signature analysis, effector T cell signature analysis, antigen presentation signature analysis, and angiogenic signature analysis.

In some embodiment, the method further comprises correlating the ITH index of each of the plurality of parameters with at least one ccRCC evolutionary subtype.

In some embodiments of the method, the at least one ccRCC evolutionary subtype comprises a VHL wildtype, a VHL monodriver, multiple clonal driver, a BAP1 driver, or PBRM1 driven tumors. In some embodiments of the method, the at least one ccRCC evolutionary subtype is selected from the group consisting of a VHL wildtype, a VHL monodriver, multiple clonal driver, a BAP1 driver, and PBRM1 driven tumors.

In some embodiments of the method, the PBRM1 driven tumors comprise PBRM1→SETD2, PBRM1→SCNA, and PBRM1→PI3K. In some embodiments of the method, the PBRM1 driven tumor is selected from the group consisting of PBRM1→SETD2, PBRM1→SCNA, and PBRM1→PT3K.

In some embodiments of the method, the VHL monodriver and multiple clonal driver subtypes correlate with an ITH index that is lower than the second median value.

In some embodiments of the method, PBRM1 driven tumors, SETD2 mutations, loss of heterozygosity (LOH) in Human Leukocyte Antigen (HLA), and loss of CDKN2A/B copy number correlates with an ITH index that is higher than the second median value.

In some embodiments of the method, PBRM1 driven tumors are associated with elevated HERV expression.

In some embodiments, the method further comprises ascertaining neoantigen heterogeneity by counting 8-11 amino acids length neoantigens in the plurality of regions within the biopsy sample. In some embodiments, the method further comprises ascertaining neoantigen heterogeneity by counting 4-8 amino acids length neoantigens in the plurality of regions within the biopsy sample. In some embodiments, the method further comprises ascertaining neoantigen heterogeneity by counting 6-10 amino acids length neoantigens in the plurality of regions within the biopsy sample. In some embodiments, the method further comprises ascertaining neoantigen heterogeneity by counting 8-12 amino acids length neoantigens in the plurality of regions within the biopsy sample. In some embodiments, the method further comprises ascertaining neoantigen heterogeneity by counting 10-14 amino acids length neoantigens in the plurality of regions within the biopsy sample. In some embodiments, the method further comprises ascertaining neoantigen heterogeneity by counting 12-16 amino acids length neoantigens in the plurality of regions within the biopsy sample.

In some embodiments of the method, an ITH index that is higher than the second median value is associated with higher neoantigen editing in the plurality of regions within the biopsy sample.

In some embodiments of the method, an ITH index that is lower than the second median value is associated with lower neoantigen editing in the plurality of regions within the biopsy sample.

In some embodiments, the method further comprises administering ICB treatment to the at least one patient and ascertaining neoantigen heterogeneity in the plurality of regions within the biopsy sample prior to and after administering the ICB treatment.

Non-limiting examples of checkpoint inhibitors include Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

In some embodiments of the method, ascertaining neoantigen heterogeneity comprises counting 8-11 amino acids length neoantigens prior to and after administering the ICB treatment. In some embodiments of the method, ascertaining neoantigen heterogeneity comprises counting 4-8 amino acids length neoantigens prior to and after administering the ICB treatment. In some embodiments of the method, ascertaining neoantigen heterogeneity comprises counting 6-10 amino acids length neoantigens prior to and after administering the ICB treatment. In some embodiments of the method, ascertaining neoantigen heterogeneity comprises counting 8-12 amino acids length neoantigens prior to and after administering the ICB treatment. In some embodiments of the method, ascertaining neoantigen heterogeneity comprises counting 10-14 amino acids length neoantigens prior to and after administering the ICB treatment. In some embodiments of the method, ascertaining neoantigen heterogeneity comprises counting 12-16 amino acids length neoantigens prior to and after administering the ICB treatment.

In some embodiments of the method, a deletion of neoantigens after administering the ICB treatment is indicative of neoantigen editing by the ICB treatment.

In some embodiments of the method, a deletion of neoantigens after administering the ICB treatment is indicative of selective pressure against hydrophobic residues and selection in favor of hydrophilic residues. In some embodiments of the method, a deletion of neoantigens after administering the ICB treatment is indicative of selective pressure against hydrophobic residues. In some embodiments of the method, a deletion of neoantigens after administering the ICB treatment is indicative of selection in favor of hydrophilic residues.

In some embodiments of the method, a deletion of neoantigens after administering the ICB treatment is indicative of selective pressure against Phenylalanine and selection in favor of Arginine and Glutamic acid. In some embodiments of the method, a deletion of neoantigens after administering the TCB treatment is indicative of selective pressure against Phenylalanine. In some embodiments of the method, a deletion of neoantigens after administering the TCB treatment is indicative of selective pressure in favor of Arginine and Glutamic acid. In some embodiments of the method, a deletion of neoantigens after administering the ICB treatment is indicative of selective pressure against one or more hydrophobic residues and selection in favor of one or more hydrophilic residues. In some embodiments of the method, a deletion of neoantigens after administering the ICB treatment is indicative of selective pressure against one or more hydrophobic residues. In some embodiments of the method, a deletion of neoantigens after administering the ICB treatment is indicative of selection in favor of one or more hydrophilic residues.

In some embodiments of the method, a deletion of neoantigens after administering the ICB treatment correlates with an ITH index that is higher than the second median value.

In some embodiments of the method, a deletion of neoantigens after administering the ICB treatment correlates with an immunosuppressive TME.

In some embodiments of the method, the ICB treatment is selected from the group consisting of Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy). In some embodiments of the method, the ICB treatment comprises Pembrolizumab (Keytruda), Nivolumab (Opdivo), Cemiplimab (Libtayo) Atezolizumab (Tecentriq), Avelumab (Bavencio), Durvalumab (Imfinzi), and lpilimumab (Yervoy). In some embodiments of the method, the ICB treatment consists of Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy). In some embodiments, other ICB treatment options are also contemplated and within the scope of the disclosure.

In some embodiments of the method, an ITH index that is higher than the second median value correlates with high myeloid signature and low effector T cell signature. In some embodiments of the method, an ITH index that is higher than the second median value correlates with high myeloid signature. In some embodiments of the method, an ITH index that is higher than the second median value correlates with low effector T cell signature. In some embodiments of the method, an ITH index that is higher than the second median value correlates with high myeloid signature or low effector T cell signature.

In some embodiments of the method, an ITH index that is higher than the second median value correlates with low antigen presentation signature.

In some embodiments of the method, an ITH index that is higher than the second median value correlates with reduced TCR diversity.

In some embodiments of the method, an ITH index that is lower than the second median value correlates with the biopsy being sparsely infiltrated by tumor infiltrating lymphocytes (TILs) and/or being infiltrated by stromal TILs. In some embodiments of the method, an ITH index that is lower than the second median value correlates with the biopsy being sparsely infiltrated by tumor infiltrating lymphocytes (TILs). In some embodiments of the method, an ITH index that is lower than the second median value correlates with the biopsy being infiltrated by stromal TILs. In some embodiments of the method, an ITH index that is lower than the second median value correlates with either the biopsy being sparsely infiltrated by tumor infiltrating lymphocytes (TILs) or the biopsy being infiltrated by stromal TILs. In some embodiments of the method, an ITH index that is lower than the second median value correlates with both the biopsy being sparsely infiltrated by tumor infiltrating lymphocytes (TILs) and the biopsy being infiltrated by stromal TILs.

In some embodiments of the method, an ITH index that is higher than the second median value correlates with the biopsy being infiltrated by substantial levels of both epithelial and stromal TILs.

In some embodiments of the method, infiltration of the biopsy by substantial levels of both epithelial and stromal TILs is correlated with an immune evasion/escape gene signature.

In some embodiments of the method, immune evasion/escape is correlated with HLA LOH and CDKN2A/B loss in the cellular fractions collected from the plurality of regions within the biopsy sample. In some embodiments of the method, immune evasion/escape is correlated with HLA LOH in the cellular fractions collected from the plurality of regions within the biopsy sample. In some embodiments of the method, immune evasion/escape is correlated with CDKN2A/B loss in the cellular fractions collected from the plurality of regions within the biopsy sample.

In some embodiments of the method, immune evasion/escape is correlated with HLA LOH in a cellular fraction collected from peripheral blood of the patient. In some embodiments of the method, immune evasion/escape is correlated with CDKN2A/B loss in a cellular fraction collected from peripheral blood of the patient.

In some embodiments of the method, the genomic analysis comprises performing small variant calling, evaluation of somatic copy number alterations, allele specific copy number calling, HLA typing, and in silico binding prediction of putative neoantigens.

In some embodiments of the method, the transcriptomic analysis comprises quantification of gene expression data, such as, for example, a gene expression microarray, RT-PCR, RNA-Seq, and the like. In a typical embodiment, the transcriptomic analysis comprises a massively parallel sequencing method such as RNA-Seq.

In some embodiments of the method, the TCR analysis comprises T cell clonotyping. Additionally, or alternatively, the TCR analysis can comprise T cell diversity estimation. Diversity estimation methods are known in the art, and include, for example, using a diversity index such as Shannon Entropy index, Simpson's Diversity index, and Berger Parker index. In a typical embodiment, the method utilizes T cell diversity estimation using Shannon Entropy Index.

In some embodiments of the method, the immune cell analysis comprises a gene signature analysis, such as, for example, a tumor microenvironment gene signature analysis. In some embodiments, the gene signature analysis comprises use of a gene set enrichment analysis, for example a single sample Gene Set Enrichment Analysis (ssGSEA) method, as is known in the art and exemplified by Barbie D A, Tamayo P, et al. Systematic RNA interference reveals that oncogenic KRAS-driven cancers require TBK1. Nalum. 2009; 462:108-112.

In some embodiments of the method, the metabolomics analysis comprises quantification of major metabolites in a tumor tissue sample. Techniques for quantification of metabolites from a tissue sample are known to those of skill in the art and include liquid chromatography-mass spectrometry (LC-MS) and tandem mass spectroscopy (MS/MS).

In some embodiments of the method, the pathology analysis comprises tumor-stroma-immune grading using a pathology method as is known in the art, such as, for example, methods to classify tumors to N-TIL (tumors sparsely infiltrated by TILs), S-TIL (tumors dominated by stromal TILs), and ES-TIL (tumors with substantial levels of both epithelial and stromal TILs).

In some embodiments of the method, the myeloid signature analysis comprises ssGSEA analysis to evaluate enrichment of one or more of the following genes: IL6, CXCL1, CXCL2, CXCL3, CXCL8, and PTGS2.

In some embodiments of the method, the JAVELIN signature analysis comprises ssGSEA analysis to evaluate enrichment of one or more of the following genes: NRARP, NRXN3, CALCRL, TEK, ECSCR, PTPRB, CD34, RAMP2, KDR, NOTCH4, FLT1, GJA5, TBX2, HEY2, ARHGEF15, SMAD6, AQP1, GATA2, ENPP2, ATP1A2, EDNRB, VIP, KCNAB1, RAMP3, CACNB2, and CASQ2.

In some embodiments of the method, the effector T cell signature analysis comprises ssGSEA analysis to evaluate enrichment of one or more of the following genes: CD8A, EOMES, PRF1, IFNG, and CD274.

In some embodiments of the method, the antigen presentation signature analysis comprises ssGSEA analysis to evaluate enrichment of one or more of the following genes: HLA-A, HLA-B, HLA-C, B2M, TAP1, TAP2, and TAPBP.

In some embodiments of the method, the angiogenic signature analysis comprises ssGSEA analysis to evaluate enrichment of one or more of the following genes: VEGFA, KDR, ESM1, PECAM1, ANGPTL4, and CD34.

In some embodiments of the method, the immune evasion/escape gene signature comprises ssGSEA analysis to evaluate enrichment of one or more of the following genes: TIMP1, PXDN, COL15A1, OLFML2B, COL5A2, DLX5, SOX11, KLHDC8A, UNC5A, ADAMTS14, MMP11, and FN1.

In some embodiments, cell-type specific markers are selected from the group consisting of: human endogenous retroviral (HERV) gene expression markers, tumor infiltrating lymphocyte (TIL) markers, microsatellite instability (MSI) status markers, and tumor mutational burden (TMB) markers.

In some embodiments, cell-type specific markers comprise markers associated with one or more of CD8+ T, CD4+ T, and CD19+ B cells.

In some embodiments, the method further comprises administering ICB treatment to the cancer patient if the ITH index is lower than the second median value.

In some embodiments, the method further comprises administering a non-ICB cancer therapy and not administering an ICB treatment to the cancer patient if the ITH index is higher than the second median value.

In some embodiments, the method further comprises administering a non-ICB cancer therapy and withholding an ICB treatment to the cancer patient if the ITH index is higher than the second median value.

### Methods of Predicting an Outcome of Immune Checkpoint Inhibitor (ICB) Therapy

In some embodiments, a method of predicting an outcome of immune checkpoint inhibitor (ICB) therapy is provided.

In some embodiments, the method comprises providing a biopsy sample from a cancer patient, ascertaining a weighted genome instability index (wGII) based on a mutational frequency in a plurality of regions within the biopsy sample, comparing the wGII to a first median value and classifying the plurality of regions within the biopsy sample as having either a higher wGII than the first median value or a lower wGII than first median value, analyzing a plurality of parameters in the plurality of regions within the biopsy sample, classified as having either higher wGII than median or lower wGII than median, to determine an ITH index of each of the plurality of parameters of each of the plurality of regions within the biopsy sample, comparing the ITH index of each of the plurality of parameters to a second median value, and classifying each of the plurality of parameters as having either higher ITH index than the second median value or a lower ITH index than the second median value. A higher ITH index than the second median value is predictive of the patient being nonresponsive to ICB therapy. A lower ITH index than the second median value is predictive of the patient being responsive to ICB therapy.

In some embodiments of the method, the cancer is clear cell renal cell carcinoma (ccRCC).

In some embodiments of the method, the biopsy sample is a nephrectomy sample.

In some embodiments, other cancers are also contemplated and within the scope of the disclosure. Non-limiting examples include selected from the group consisting of colorectal (CRC), breast adenocarcinoma, pancreatic adenocarcinoma, lung carcinoma, prostate cancer, glioblastoma multiform, hormone refractory prostate cancer, solid tumor malignancies such as colon carcinoma, non-small cell lung cancer (NSCLC), anaplastic astrocytoma, bladder carcinoma, sarcoma, ovarian carcinoma, rectal hemangiopericytoma, pancreatic carcinoma, advanced cancer, cancer of large bowel, stomach, pancreas, ovaries, melanoma, pancreatic cancer, colon cancer, bladder cancer, hematological malignancies, squamous cell carcinomas, breast cancer, glioblastoma, brain neoplasms, pilocytic astrocytoma, diffuse astrocytoma, anaplastic astrocytoma, brain stem gliomas, glioblastomas multiforme, meningioma, ependymomas, oligodendrogliomas, mixed gliomas, pituitary tumors, craniopharyngiomas, germ cell tumors, pineal region tumors, medulloblastomas, and primary CNS lymphomas.

In some embodiments of the method, the biopsy sample is a sample from colorectal (CRC), breast adenocarcinoma, pancreatic adenocarcinoma, lung carcinoma, prostate cancer, glioblastoma multiform, hormone refractory prostate cancer, solid tumor malignancies such as colon carcinoma, non-small cell lung cancer (NSCLC), anaplastic astrocytoma, bladder carcinoma, sarcoma, ovarian carcinoma, rectal hemangiopericytoma, pancreatic carcinoma, advanced cancer, cancer of large bowel, stomach, pancreas, ovaries, melanoma, pancreatic cancer, colon cancer, bladder cancer, hematological malignancies, squamous cell carcinomas, breast cancer, glioblastoma, brain neoplasms, pilocytic astrocytoma, diffuse astrocytoma, anaplastic astrocytoma, brain stem gliomas, glioblastomas multiforme, meningioma, ependymomas, oligodendrogliomas, mixed gliomas, pituitary tumors, craniopharyngiomas, germ cell tumors, pineal region tumors, medulloblastomas, and primary CNS lymphomas.

In some embodiments of the method, analyzing the plurality of parameters in the plurality of regions within the biopsy sample is performed by at least one of collecting DNA, RNA, cellular fraction, tissue section, and tissue extraction for each of the plurality of regions within the biopsy sample.

In some embodiments of the method, the plurality of parameters comprises genomic analysis, transcriptomic analysis, TCR analysis, immune cell analysis, metabolomics analysis, pathology analysis, myeloid signature analysis, JAVELIN signature analysis, effector T cell signature analysis, antigen presentation signature analysis, and angiogenic signature analysis.

In some embodiments of the method, the plurality of parameters is selected from the group consisting of genomic analysis, transcriptomic analysis, TCR analysis, immune cell analysis, metabolomics analysis, pathology analysis, myeloid signature analysis, JAVELIN signature analysis, effector T cell signature analysis, antigen presentation signature analysis, and angiogenic signature analysis.

In some embodiments of the method, the plurality of parameters comprises two or more of genomic analysis, transcriptomic analysis, TCR analysis, immune cell analysis, metabolomics analysis, pathology analysis, myeloid signature analysis, JAVELIN signature analysis, effector T cell signature analysis, antigen presentation signature analysis, and angiogenic signature analysis.

In some embodiments of the method, the ICB treatment is selected from the group consisting of: Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy). In some embodiments of the method, the ICB treatment comprises Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy). In some embodiments of the method, the ICB treatment consists of Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy). In some embodiments, other ICB treatment options are also contemplated and within the scope of the disclosure.

In some embodiments, the method further comprises administering ICB treatment to the cancer patient if the ITH index is lower than the second median value.

In some embodiments, the method further comprises administering a non-ICB cancer therapy and not administering an ICB treatment to the cancer patient if the ITH index is higher than the second median value.

In some embodiments, the method further comprises administering a non-ICB cancer therapy and withholding an ICB treatment to the cancer patient if the ITH index is higher than the second median value.

### EXAMPLES

The following examples are non-limiting and other variants within the scope of the art also contemplated.

### Exanple 1—Intra-Tumoral Heterogeneity in Response to ICB in ccRCC Patients

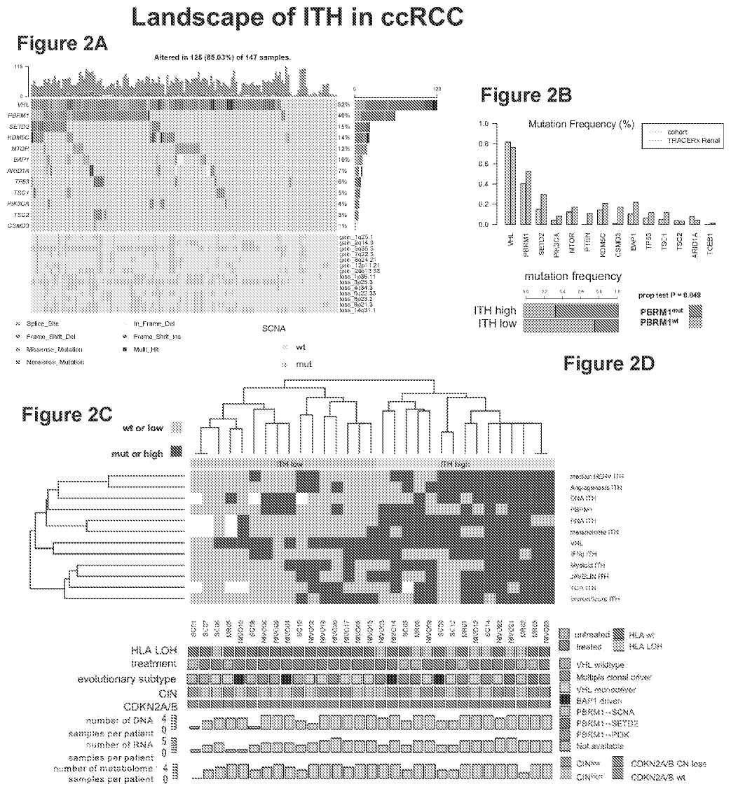

Previous studies have demonstrated ITH in ccRCC using multi-regional sequencing (M-seq) (Gerlinger et al., 2014). Notably, (Gerlinger et al., 2014) found that nearly 75% of mutations seen in ccRCC seems to be subclonal which can potentially confound the estimates of driver mutation prevalence. However, one potential limitation of this study might be attributed to a relatively low coverage of M-seq which can lead to overestimation of ITH. M-seq can remove this barrier and facilitates studying ITH as well as tumor clonal evolution. In some embodiments, M-seq analysis is utilized this approach to study TME heterogeneous response to therapy across different regions of tumor. Using ultra-deep (median coverage of 360X) multi-regional WES (MWES), in some embodiments, the mutational profile of 147 biopsies across 31 patients is portrayed. As expected, VHL and PBRM1 mutations as well as loss of 3p25 cytoband were most frequently observed in this cohort (FIG. 2A). The frequency of different genomic alterations was in agreement with a previous MWES study by TRACERx Renal (FIG. 2B) (Turajlic et al., 2018b). Moreover, the SNV/Indel mutations were validated by MSK-IMPACT results for those patients where the data was available.

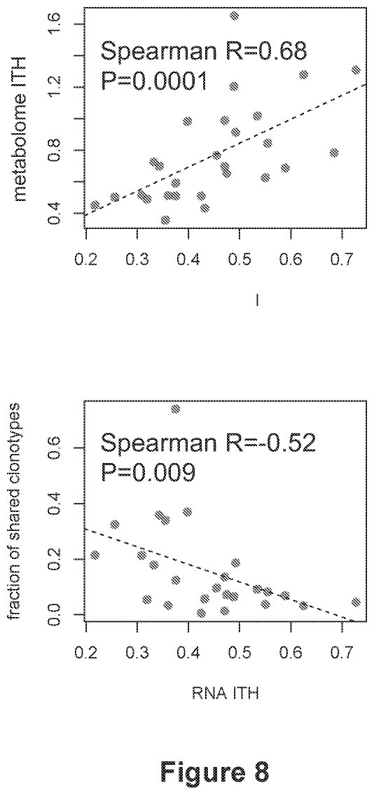

While mutational heterogeneity has been well described, its relationship to transcriptional, metabolic and microenvironment heterogeneity has yet to be elucidated. To address this, in some embodiments, the inventors integrated the M-seq data with ITH metrics across multi-omics platforms. Accordingly, we defined an ITH index per data type including DNA, RNA, and metabolome (See, Examples 8-28). Interestingly, we identified a high correlation between ITH measured by WES, whole transcriptomics (WTS) and metabolomics (FIG. 8). Using M-seq (Turajlic et al., 2018b) demonstrated ITH and weighted genome instability index (wGII) are associated with distinct ccRCC evolutionary subtypes when patients are classified as ITH high versus low and wGII high versus low using median to define high and low. Following this approach, in order to evaluate the interaction between ITH of different features, we first classified each feature as high ITH versus low ITH using median as a cut-off and used unsupervised hierarchical clustering to reveal potential ccRCC subgroups (FIG. 2C). Intriguingly, we observed two distinct subtypes enriched in low and high ITH across different data types confirming ITH and heterogenous response can be seen across-omics features (See, Examples 8-28).

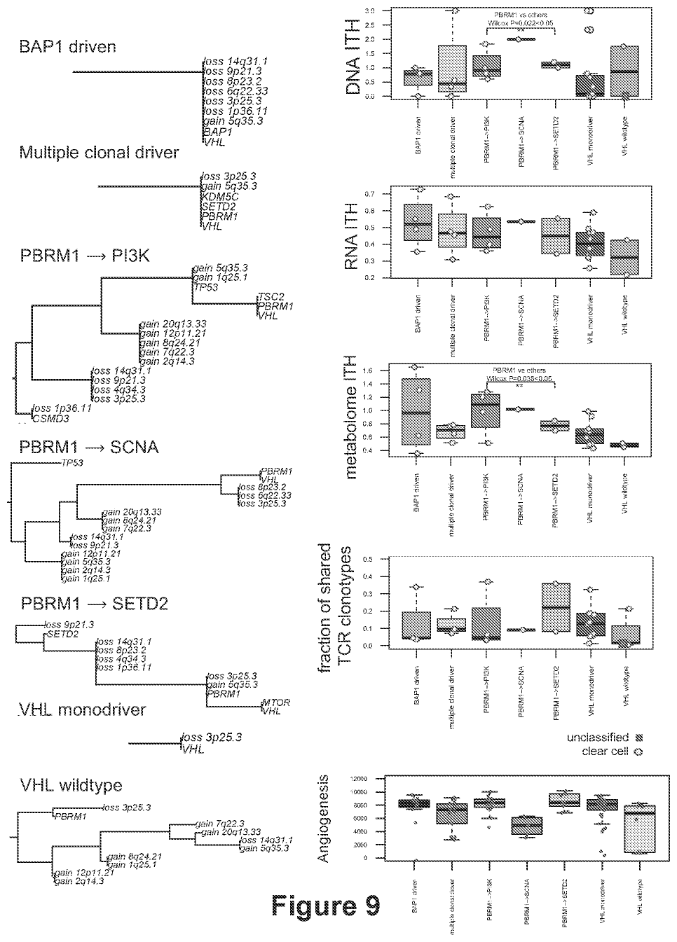

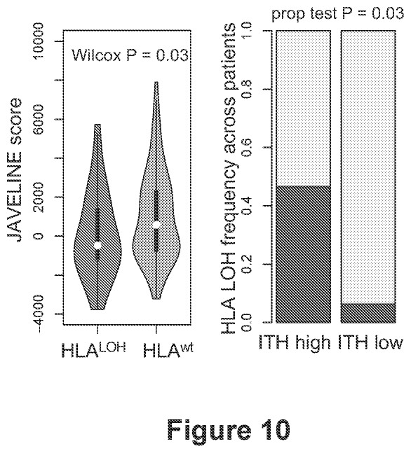

Different factors intrinsic to tumor or TME can contribute to ITH (Turajlic et al., 2018b) and tumor clonal evolution. In TRACERx Renal (Turajlic et al., 2018b), researchers uncovered distinct evolutionary subtypes with distinct genomic properties as well as diverse clinical outcomes (Turajlic et al., 2018a). ccRCC evolutionary subtypes comprise VHL wildtype, VHL monodriver, multiple clonal driver (defined as patients with at least 3 clonal driver mutations), BAP1 driven, and finally PBRM1 related tumors which consists of PBRM1→SETD2 (presence of both PBRM1″″ and SETD2″″), PBRM1→SCNA (presence of PBRM1″″ and driver somatic copy number alteration), and PBRM1→PI3K (presence of PBRM1″″ and any mutations related to PI3K pathway). We classified the patients in our study to these evolutionary subtypes (FIG. 9) and observed while low ITH is a property of VHL monodriver and multiple clonal driver subtypes, PBRM1 driven tumors tend to exhibit a high ITH in agreement with TRACERx Renal (Turajlic et al., 2018b). Incorporating ccRCC evolutionary subtypes into our model (FIG. 2C), we further illustrated ITH associated with PBRM1 can be inferred across different measurements of ITH suggesting PBRM1 as a major contributor to the observed ITH (FIG. 2D). Similarly high angiogenic activity is a distinct feature of PBRM1→SETD2 and PBRM1→PI3K but not PBRM1→SCNA as shown in FIG. 9. ITH high tumors were also enriched for SETD2 mutations in agreement with (de Matos et al., 2019) (although did not reach statistical significance due to smaller sample size), loss of heterozygosity (LOH) in Human Leukocyte Antigen (HLA) and CDKN2A/B copy number loss (FIGS. 2C and 10). HLA LOH can be associated with reduced clinical benefit of ICB treatment through immune evasion (Chowell et al., 2018, McGranahan et al., 2017) in lung cancer. HLA LOH was observed in 9 out of 33 (27%) patients with WES data available (7 out of the 31 patients whose WTS and metabolomics data were also available). We noted that 7 out 9 incidents of HLA LOH were subclonal. This was in agreement with a previous study (McGranahan et al., 2017) in lung cancer. Low occurrence of a clonal HLA LOH further highlights the limited sensitivity of single regional WES where only 2 out of 33 patients (6%) could be estimated to experience this HLA alteration. We observed a strong association between HLA LOH and ITH with 8 patients (21 samples) out 15 ITH high patients harbored HLA LOH while this alteration was detected in only one ITH low patient (patient level prop test P=0.07, sample level prop test P=0.0007). Notably HLA LOH in this ITH low patient was detected in all 4 regions (i.e. clonal HLA LOH) and this patient was also resistant to ipi/nivo immunotherapy as shown in our previous study (Krishna et al., 2021).

Moreover, HLA LOH was also associated with the lack of inflammatory response in ICB treated ccRCC patients (FIG. 10). This association remains strong even when multiple regions of the same patient were assessed suggesting that HLA LOH plays a crucial role in eliciting the previously seen heterogenous immunological response in several patients. However, we did not observe any association between the fraction of genome altered (referred to copy number unstable CIN^{high} vs CIN^{low}) and ITH. Together, our results demonstrate that ITH is correlated across omic strata, correlates with specific genomic events (ie PBRM1 mutations, HLA LOH) and is associated with an myeloid inflammatory landscape.

### Example 2—the Landscape of Heterogeneity of Neoantigen Editing in RCC

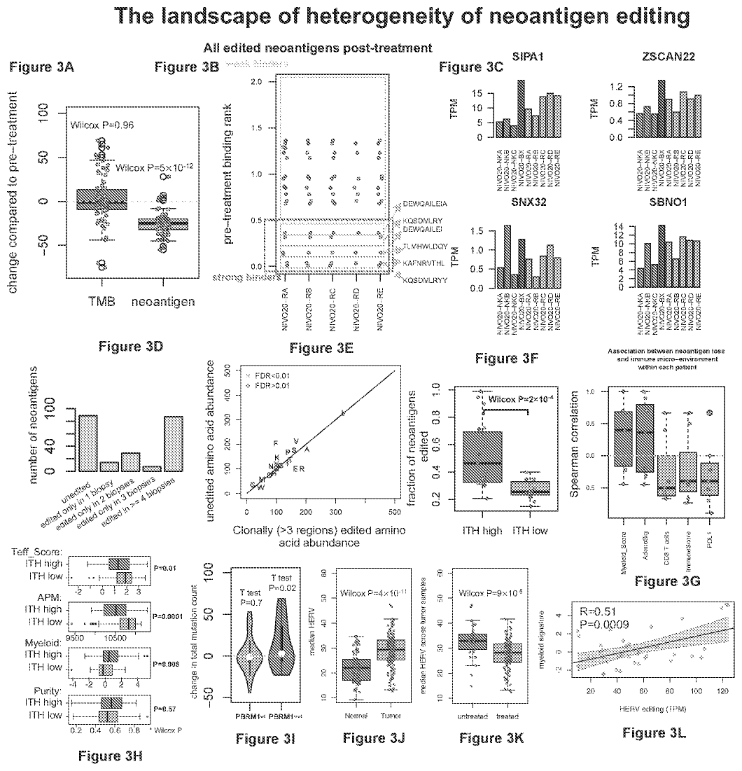

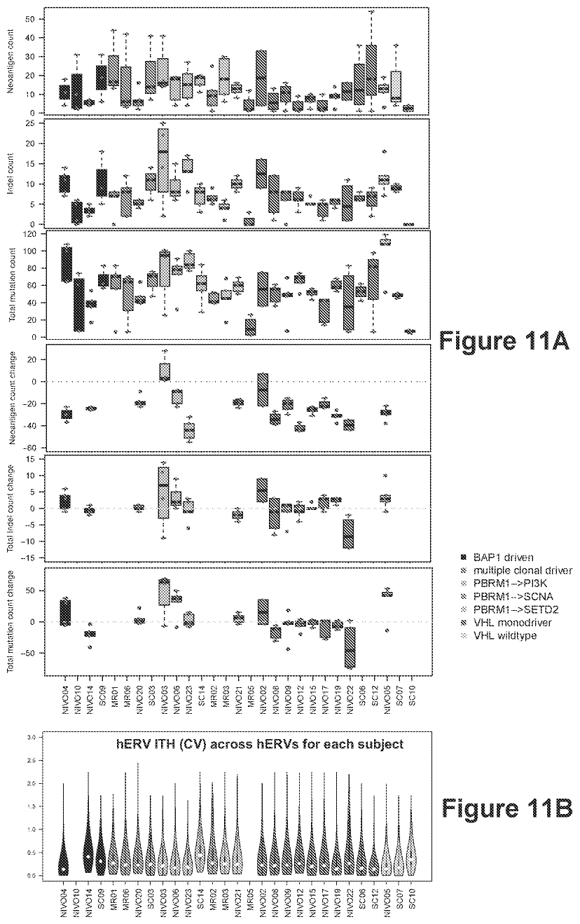

Immune-evasion has been previously described in NSCLC (Rosenthal et al., 2019). However, the impact of ICB treatment on mutation load and immune editing in ccRCC is not well characterized. A search for an association between mutational load and previously described evolutionary subtypes; however, no relation between any of the subtypes and SNV or indel driven mutations or neoantigens were observed (FIGS. 11A and 11B). Next by including all patients whose WES and WTS were performed on their pre-treatment biopsies (16 patients), the change in mutation and neoantigens upon ICB treatment (neoadjuvant Nivolumab) was assessed. Notably no trend in the change of either SNV or indel mutational count was observed. On the contrary, neoantigen counts were consistently reduced across all patients and all biopsies with the exception of NIVO03 (PBRM1→PI3K) (FIGS. 11A, 11B, and 3A).

Without being limited by any particular theory, the reduction in neoantigens can represent the impact of cytotoxic immune cells on expressed neoantigen upon ICB treatment and therefore, loss of neoantigens due to the death of neoantigen expressing cancer cells. Alternatively, neoantigen reduction may be due to selective pressure to delete more immunogenic neoantigens through either of the previously described mechanisms including reduced expression, copy number loss or gene promoter methylation (Rosenthal et al., 2019).

In order to characterize the heterogeneity of neoantigen editing, some embodiments focused on patient NIVO20 and counted all 8-11 amino acids length neoantigens seen prior to treatment but were deleted in at least one biopsy after treatment. Interestingly, all identified 6 edited neoantigens were deleted in at least 4 regions suggesting neoantigen editing is a clonal event (FIG. 3B). This was further confirmed by evaluation of the expression of the genes producing these edited neoantigens such that all regions experienced a 2-3-fold reduction in the expression of these genes (FIG. 3C). The finding was also confirmed on all patients whose both pre-treatment and post-treatment regions were available (FIG. 3D). To determine whether a selective pressure exists on certain neoantigens, in some embodiments, the number of amino acids preserved versus edited upon immunotherapy was compared, which showed a strong selection against Phenylalanine (F, extremely hydrophobic)—in favor of Arginine (R, extremely hydrophilic) and Glutamic acid (E, extremely hydrophilic) in our cohort (FIG. 3E) suggesting a selective pressure against hydrophobic residues which tends to be more immunogenic (Riley et al., 2019).

We next quantified the degree of neoantigen editing by measuring the average number of neoantigens deleted per biopsy (fraction of neoantigens edited) i.e. the ratio of the deleted neoantigens in a treated region compared to pre-treatment over the total number of pre-treatment neoantigens. Note that the fraction of neoantigens edited may not correlate with the change in the neoantigen counts since each regional biopsy may possess distinct mutations some of which can be immunogenic neoantigens upon treatment as part of tumor evolution. Comparing the fraction of neoantigens edited between our previously identified subtypes, we observed a significantly higher degree of editing in the ITH high subtype (FIG. 3F).

Recent advances in immune deconvolution methods allow for assessment of microenvironmental response using bulk transcriptional data. Moreover, several TME gene expression signatures correlate with ccRCC patients' response to ICBs and other antiangiogenic therapies such as myeloid signature (McDermott et al., 2018), JAVELIN signature (Motzer et al., 2020b), and angiogenesis signature (See, Examples 8-28). Strikingly, the fraction of neoantigens edited was associated with myeloid high TME among patients. Likewise, the correlation between the degree of neoantigen editing and immune-suppressive TME was also noticeable even within each patient with high editing regions depicting the highest myeloid and lowest ImmuneScore in most patients (FIG. 3G). Strikingly, we observe that ITH high tumors (defined as all regions belonging to a patient who is classified as ITH high) were enriched with an immune-suppressive immune-phenotype as measured by high myeloid and low T cell effector (Teff) signatures (FIG. 3H). Besides, a signature associated with antigen presentation (APM) was downregulated in ITH high patients consistent with other APM defects such as HLA LOH in the ITH high subtype. However, no association between tumor purity and ITH was observed suggesting that our findings are not confounded by tumor purity.

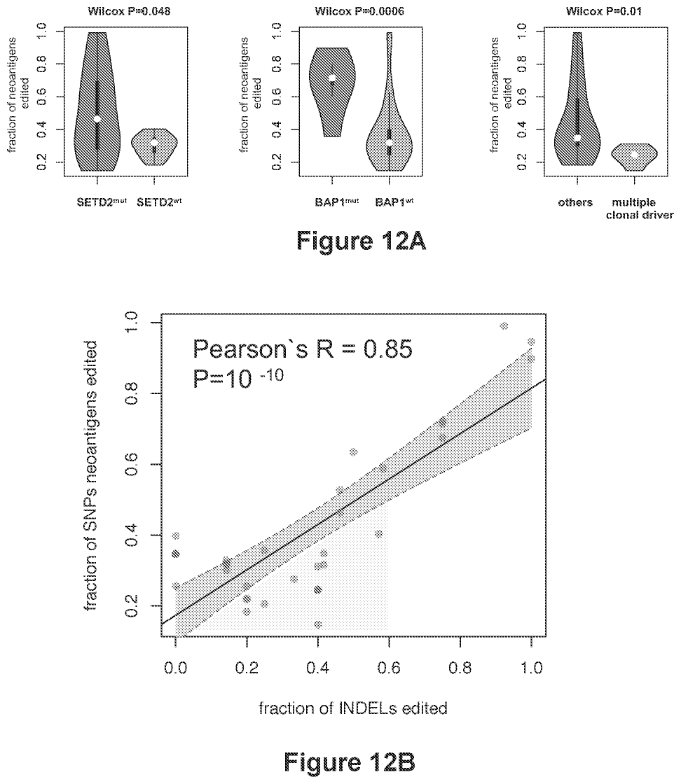

We noticed that the total mutational counts increase in several PBRM1 mutant patients compared to PBRM1 wildtype (FIG. 3I). This may imply that tumors can accumulate more mutations as a natural part of cancer evolution which may lead to an increase in total mutation count over time but these tend to be less immunogenic (i.e. non-neoantigen) as supported by a consistent decrease in neoantigens across patients. (FIGS. 11A and 5A). Lastly, in addition to SNPs derived neoantigens, INDELs have been shown to be a strong source of immunogenic neoantigens. In our study, we observed an association between neoantigen depletion and ccRCC driver mutations (FIG. 12A) and a strong correlation between the fraction of INDELs edited and the fraction of neoantigens (SNPs only) edited while a trend towards higher INDEL depletion was noted in several patients (FIG. 12B). All in all, these findings are consistent with several lines of evidence (Jonasch et al., 2020) that both PBRM1 and high ITH (Williams et al., 2020) are associated with lack of IFN-γ production by reducing IFNγ-JAK2-STAT1 signaling (Liu et al., 2020).

### Example 3—HERV Derived Neoantigen Editing in ccRCC

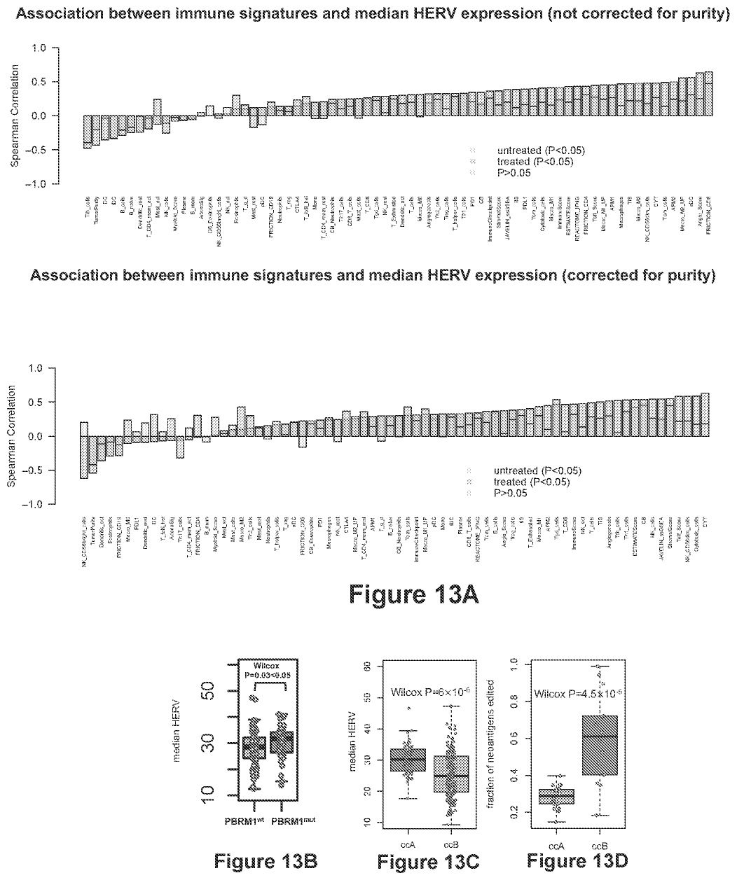

Human endogenous retroviral (HERV), a family of retrotransposons constitutes about 1-8% of the human genome (Nelson et al., 2003), and has been proposed to be highly expressed in ccRCC and associated with ICB response (Smith et al., 2019). Hence, we looked to elucidate whether immunoediting can be observed across HERV derived potential antigenic peptides. Previous study by (Braun et al., 2020) questioned whether WTS may not render the accurate quantification of HERV expression possible. However, the lack of association between HERV expression (as quantified by WTS) in (Braun et al., 2020) study might be due to lower WTS depth. To overcome this we performed Deep WTS (Golkaram et al., 2021) enabling us to quantify HERV expression with a higher limit of detection (&e, Examples 8-28, ˜200 million paired reads per library). Therefore, we employed our previously established method to accurately estimate the genome wide expression of HERVs in our cohort (Golkaram et al., 2021). First, we confirmed the overexpression of HERVs in tumors compared to normal tissues in our cohort (FIG. 3J, median of all HERV loci investigated is shown). Comparing the expression of HERVs between untreated and treated samples, we noticed a substantial reduction in HERVs, an observation akin to the change to SNV/Indel driven neoantigens (FIG. 3K). This was further confirmed by a high correlation between the median expression of different HERV loci and TIL abundance (FIG. 13A). This observation remained valid even when HERV expression was corrected for tumor purity (FIG. 13A), and also was confirmed in our previous study (Golkaram et al., 2021). Likewise, we observed a strong correlation between HERV editing (i.e., change in the expression of immunogenic HERV loci after treatment, See, Examples 8-28) and myeloid signature further highlighting the association between neoantigen depletion and myeloid activation (FIG. 3L). Overall, our findings point to HERVs as a potential target of immune surveillance in ccRCC.

The mechanism of HERV activation is still an active area of investigation; however, demethylation of HERVs promoters as well as hypoxia have shown to contribute (Chiappinelli et al., 2015; Choueiri and Kaelin, 2020). Notably, HIF1a has been proposed as a HERV associated transcription factor (Cherkasova et al., 2011). HIF1a activity is repressed by VHL, a tumor suppressor. Therefore, we hypothesized a combination of HERV promoter demethylation together with VHL inactivation should contribute to HERV over expression. Accordingly, we observed a high correlation between angiogenic signatures and median HERV confirming this hypothesis (FIG. 13A). Likewise, PBRM1 mutations were also positively associated with HERV expression aligned with the positive association described between high angiogenic activity and both HERV activation and PBRM1 mutation (FIG. 13B). Finally, we assess the HERV expression with the prognostic ccA and ccB ccRCC classifier (Ghatalia and Rathmell, 2018). ccA tumors are noted to be more indolent and have high angiogenic expression (Hakimi Cancer disc). Consistently, ccA tumors demonstrated a substantially higher expression of HERVs (FIG. 13C). Strikingly, ccB subtype which is associated with poor prognosis (Ghatalia and Rathmell, 2018), also demonstrated a significantly higher immune escape compared to ccA subtype (FIG. 13D).

### Example 4—Branch Evolution Model for Immune Escape and Intra-Tumoral Heterogeneity

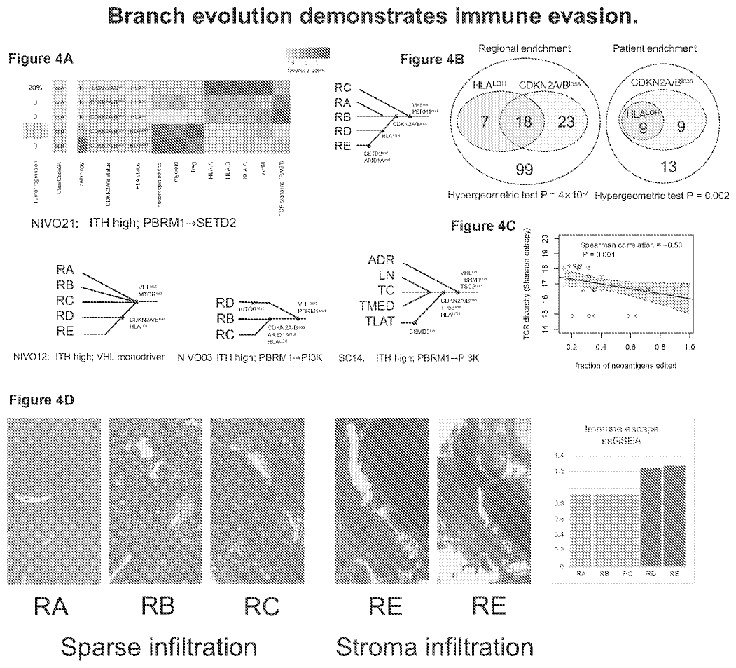

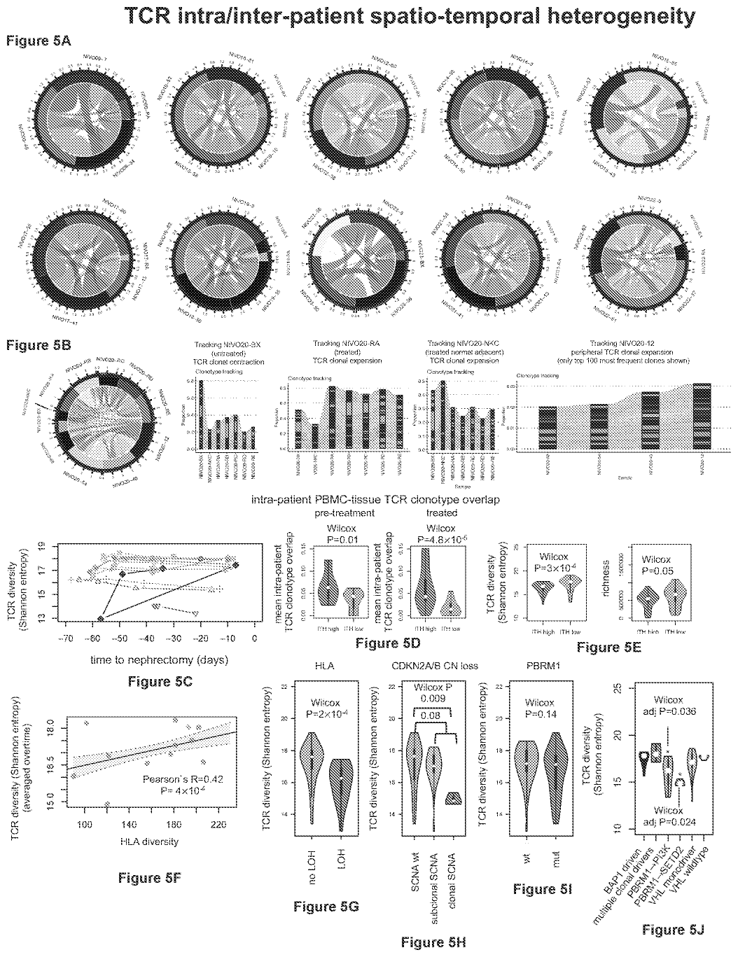

To further shed light into how tumor subpopulations exploit genomic alterations to escape immune surveillance, we focused on patients whose tumors underwent subclonal immunoediting. Strikingly, subclonal reconstruction revealed evolution of clones whose genomic alterations such as concurrent HLA LOH and CDKN2A/B loss could facilitate immune evasion in distinct regions of tumors (FIGS. 4A and 4B). As previously shown (FIG. 3G), regions with high immunoediting were also associated with myeloid activation and high Treg signature. Likewise, we observed that a reduction in T cell diversity in PBMC also correlates with immunoediting (FIG. 4C) which is in agreement with (Zhang et al., 2018) in ovarian cancer where researchers highlighted a link between T cell clonality and neoantigen depletion.

Histopathologic evaluation of the tumor by a dedicated genitourinary pathologist revealed a distinct TME pertaining to neoantigen depletion and ICB resistance (FIG. 4D). Namely, we discovered that regions with CDKN2A/B alteration and HLA LOH represent tumor infiltrating lymphocytes (TILs) resident within the stroma. These findings suggest that despite abundant TILs, lack of cancer cell-lymphocyte colocalization and reduced tumor-immune engagement may result in a failure of immune recognition or region-specific barriers to infiltration.

### Example 5—TCR Intra/Inter-Patient Heterogeneity

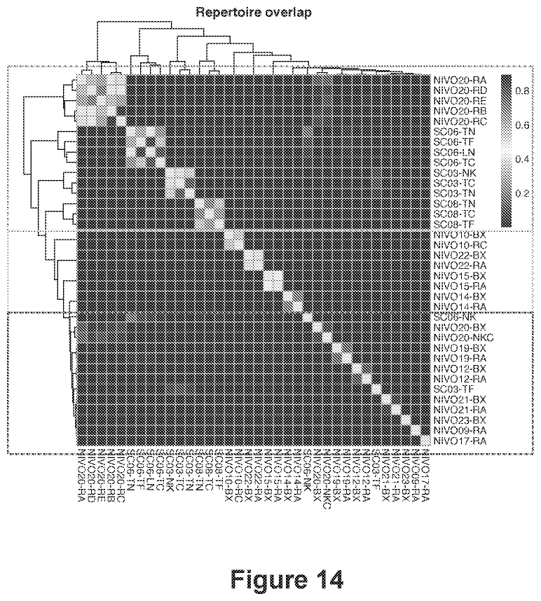

T cell receptor (TCR) ITH has been described in several studies and has been linked to intratumoral ITH in lung cancer (Reuben et al., 2017; Zhang et al., 2016). Repertoire overlap analysis (FIGS. 5A and 14) illustrated a high degree of shared clonotypes across different regions but a lack of shared clonotypes across patients. Leveraging serially collected PBMC derived T cell clones (See, Examples 8-28), we observed only 1-15% PBMC TCR overlap with tissue resident clones. Further, roughly 10-60% of PBMC T cell clones are present at different time points throughout the course of ICB therapy highlighting the temporal heterogeneity of TCR repertoire (FIG. 5A).

ICB treatment can also influence the dominant TCR clonotypes. A study by (Wu et al., 2020) indicated that intra-tumoral T cells, especially in responsive patients, are replenished with fresh, non-exhausted replacement cells from sites outside the tumor. Focusing on patient NIVO20 whose TCR clonal information were available at all tumor regions and different time points, we investigated whether ICB therapy induces immune response by attracting fresh T cell clones. Although we observed a significant overlap between TCR clones before and after ICB treatment, therapy expanded several novel dominant TCR clones while contracted exhausted T cell clones both residing in PBMC and tissue (FIG. 5B). By measuring TCR repertoire diversity using Shannon's entropy, we observed a significant difference in PBMC T cell diversity among different patients; however, TCR diversity did not vary significantly during therapy. This can be explained by concomitant expansion and contraction of several distinct clones which alleviates the overall temporal variation in TCR diversity (FIG. 5C).

Next to pinpoint any associations between ITH and TCR repertoire, we compared the overlap between tissue and PBMC resident T cells among these two groups and observed a significantly higher overlap between tissue resident and PBMC derived T cells in patients with high ITH both prior and post-therapy (FIG. 5D). Likewise, ITH low patients demonstrated a significantly higher peripheral TCR diversity compared to ITH high patients (FIG. 5E). To assess whether T cell diversity is linked to antigen presentation, we investigated the correlation between both germline HLA diversity and allele specific HLA loss and observed a strong positive association between both HLA diversity and the lack of somatic alterations in HLA region (FIGS. 5F and 5G). In addition, clonal CDKN2A/B loss was associated with reduced peripheral TCR diversity (FIG. 5H). Moreover, two PBRM1 driven evolutionary subtypes with higher ITH (but not PBRM1 mutation itself) were also associated with low peripheral TCR diversity (FIGS. 51 and 5J). Together, our results demonstrate ITH associated factors can jeopardize immune activation and result in a reduced peripheral T cell diversity.

### Example 6—Immune Escape Correlates with Stroma and Myeloid Signatures

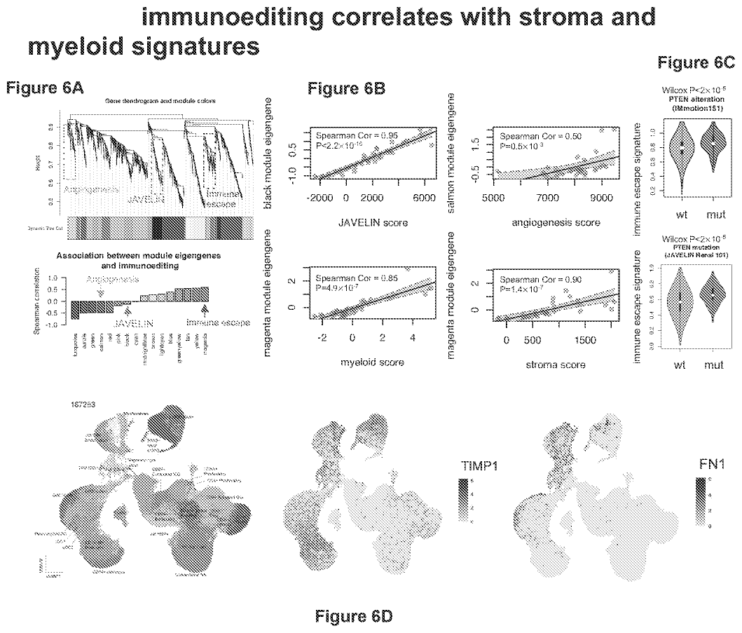

Next, in order to reveal the immunophenotype associated with adverse immunoediting, we performed Weighted Gene Coexpression Network Analysis (WGCNA) (Langfelder and Horvath, 2008) to reconstruct modules from our transcriptomic samples similar to (Motzer et al., 2020b) (FIG. 6A). Notably, as described by (Motzer et al., 2020b) we identified two modules, black and salmon which reflect immune inflammatory response (JAVELIN signature) and angiogenesis (FIGS. 6A and 6B). A previous study indicated that subclonal contraction i.e., a decrease in cancer cell fraction of immunogenic neoantigens is associated with anti-tumor immunity. Thus, to distinguish between the fraction of neoantigens edited due to immune escape but not genomic contraction, we excluded any depleted neoantigens without a co-occuring escape mechanism i.e. lack of HLA LOH or reduced neoantigen expression (See, Examples 8-28). Strikingly, module Magenta was strongly associated with neoantigen depletion as demonstrated by the high correlation between the module eigengene and the fraction of neoantigens edited (due to immune escape) per sample while no association with JAVELIN or angiogenesis signature was observed (FIG. 6A). Correlation analysis with previously known gene expression signatures illustrated that our immune escape derived gene signature is strongly associated with myeloid and stroma features of TME. Moreover, this signature is also in agreement with a pan-cancer TGFβ signature derived in a previous study (Chakravarthy et al., 2018) where they also established a link between cancer-associated fibroblasts to immune evasion and immunotherapy failure. A recent pan-tumor study (Montesion et al., 2021) highlighted co-occurrence of several genetic alterations with HLA LOH and immune evasion, namely, PTEN alterations (including SCNA and small variants) which has been linked to resistance to ICB therapy. As expected, PTEN alteration was strongly associated with immune escape signature in two independent cohorts, IMmotion151 (Motzer et al., 2020a) and JAVELIN Renal 101 (only small variant calling data was available) (Motzer et al., 2020b) (FIG. 6C). Region specific enrichment of this signature (FIG. 4D) was in concordance with stroma TIL in neoantigen depleted sites.

To reveal the primary source of this signature in ccRCC TME, we leveraged scRNAseq from multiple tumor regions, lymph node, normal kidney, and peripheral blood of two ICB-naïve and four ICB-treated patients (Krishna et al., 2021) (n=167283 single cells). We identified 28 clusters (FIG. 6D) using Louvain clustering (Levine et al., 2015; Xu and Su, 2015) and each cluster is annotated based on our previous study (Krishna et al., 2021). Among 12 genes in this gene signature, only FN1 and TIMP1 expression was high enough to be detected in low pass scRNAseq data and therefore, recruited for cell type enrichment investigation. As expected, scRNAseq revealed enrichment of this signature in renal epithelium, tumor stroma as well as tumor associated macrophages (TAMs) and monocytes (FIG. 6D). Hence, scRNAseq further confirmed the association between neoantigen depletion and immune escape with stroma, renal epithelium and myeloid activation.

### Example 7—Immune Escape Predicts Clinical Outcome to ICB Therapy

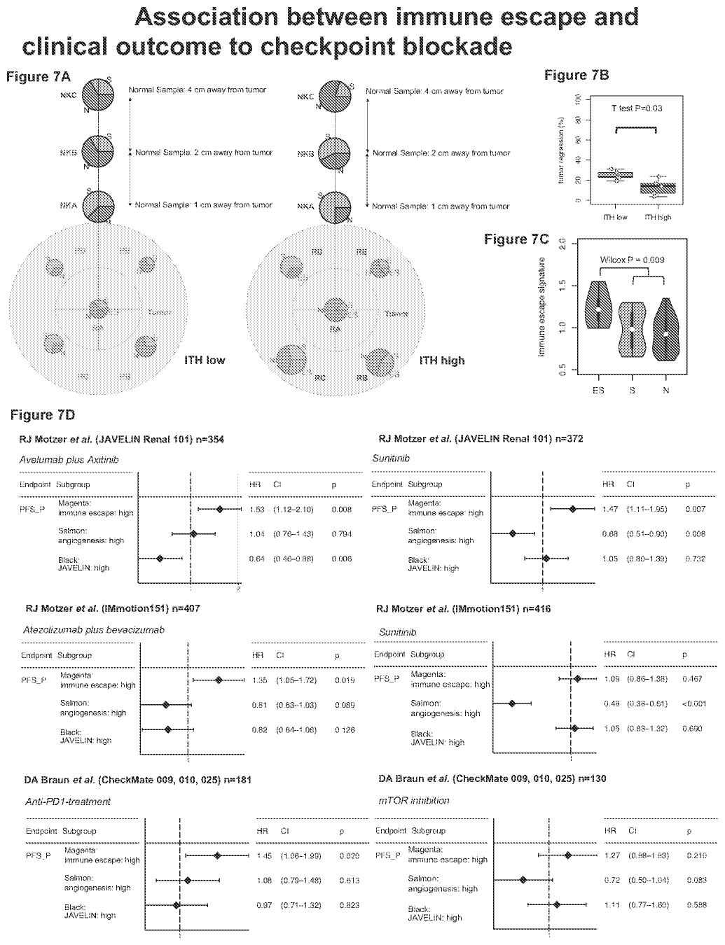

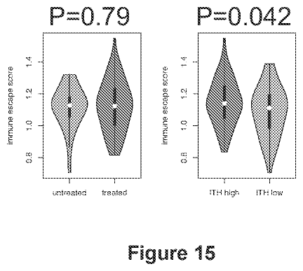

We next investigated whether ITH influences immunological response and tumor regression upon TCB treatment. As expected, comparing tumor regression after ICB treatment resulted in a significantly higher tumor regression across all regions of patients with high ITH (FIGS. 7A and 7B). (Zhang et al., 2018) revealed three major TIL subtypes: N-TIL (tumors sparsely infiltrated by TILs), S-TIL (tumors dominated by stromal TILs), and ES-TIL (tumors with substantial levels of both epithelial and stromal TILs) in ovarian cancer. Moreover, they demonstrated HLA LOH and neoantigen depletion are linked to epithelial and stroma restricted lymphocytes. Consistently, classifying our ccRCC patients to N/S/ES-TIL histologies as reviewed by an expert pathologist (where a pathology slide was available) also confirmed that an ES-TIL enriched TME is strongly associated with immune escape (FIGS. 7A and 7C). Together, this confirms that the interaction between ECM and TILs is strongly implicated in neoantigen depletion and immune escape. FIG. 15 shows boxplots of association between immune escape signature, treatment, and ITH

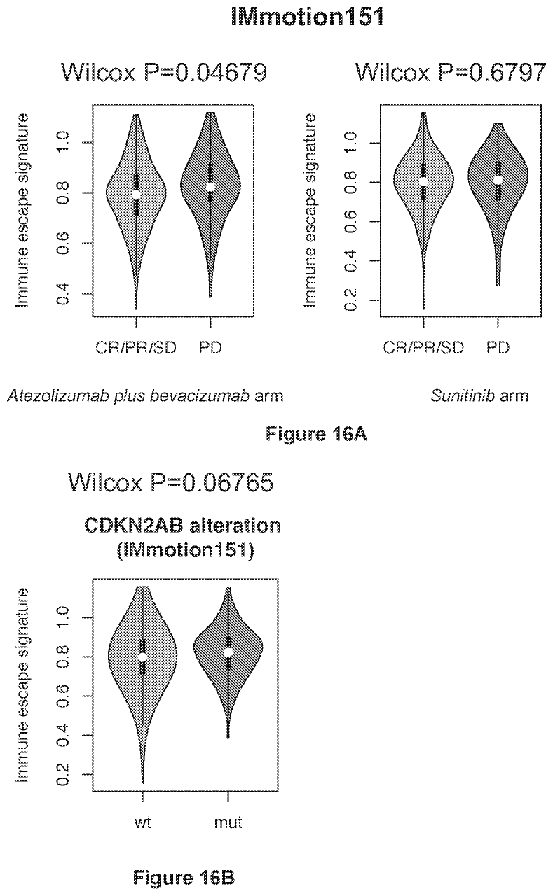

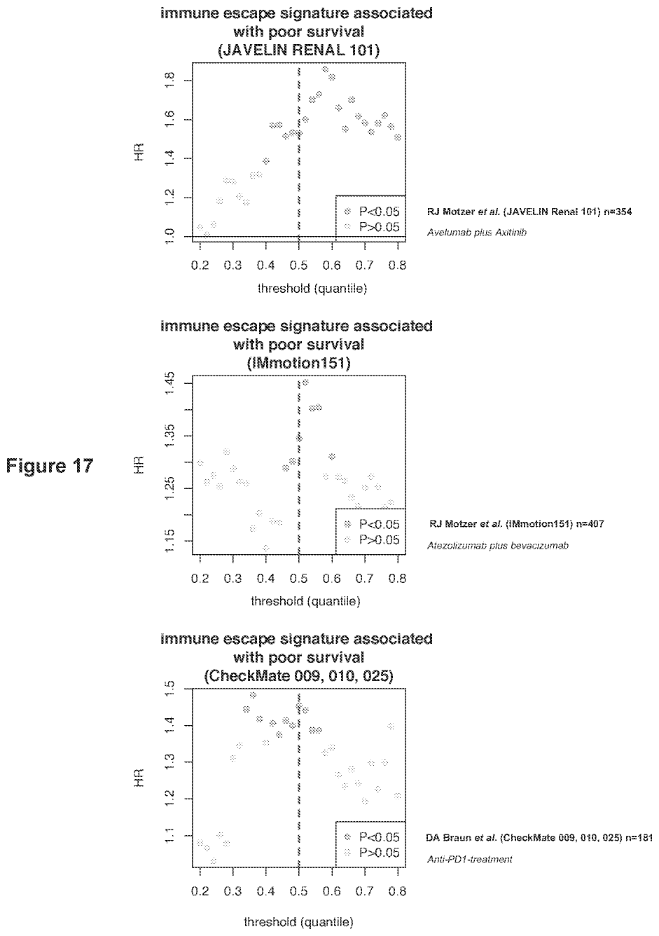

We then evaluated whether our immune escape signature can predict clinical outcome to ICB treatment. We obtained publicly available RNAseq data for several clinical trials including phase 3 JAVELIN Renal 101 trial (Motzer et al., 2020b)—a phase III randomized anti-PD-L1 (avelumab) plus tyrosine kinase inhibitor (TKI, axitinib) versus multi-target TKI (sunitinib), IMmotion151 (Motzer et al., 2020a)—a phase 3 trial comparing atezolizumab plus bevacizumab versus sunitinib in first-line metastatic renal cell carcinoma, CheckMate 009/010—a phase I/II, aPD-1 (nivolumab) treated, and CheckMate 025—a phase III randomized mTOR inhibitor (everolimus) versus aPD-1 (Braun et al., 2020). Using median score (See, Examples 8-28) of 3 gene signatures obtained in our study (i.e module salmon, black and magenta), we grouped patients into high versus low JAVELIN, angiogenic, and immune escape across different arms of each trial. JAVELIN signature was strongly predictive of clinical outcome to avelumab plus axitinib JAVELIN Renal 101 trial as reported by (Motzer et al., 2020b). However, no association with clinical benefit was found between atezolizumab plus bevacizumab or nivolumab treatment and this signature (FIG. 7D). Our angiogenesis signature was a strong predictor of response to sunitinib in both IMmotion151 and JAVELIN Renal 101 as expected. Finally, our immune escape signature was a strong predictor of response to avelumab plus axitinib, atezolizumab plus bevacizumab and nivolumab. Even though this signature was also associated with the response to sunitinib in JAVELIN Renal 101, no association between sunitinib response or mTOR inhibition was observed in IMmotion151 and CheckMate 025. FIGS. 16A and 16B show boxplots of validation of escape signature in independent cohorts (IMmotion151). FIG. 17 shows boxplots of relationship between escape gene signature and treatment outcome in different clinical trials. HRs are calculated for each threshold for ICB or ICB in combination with TKI arms in JAVELIN Renal 101, IMmotion151, and CheckMate 009, 010, 025.

### Example 8—Sample Acquisition

After acquiring informed consent and institutional review board approval from Memorial Sloan Kettering Cancer Center (MSK), partial or radical nephrectomies were performed at MSK (New York) and stored at the MSK Translational Kidney Research Program (TKRCP). Samples were flash frozen and stored at −80 degrees Celsius prior to molecular characterization. Clinical metadata was recorded for all tumor samples.

All patients represent clear cell histology and were treated via ICB or in combination with tyrosine kinase inhibitor (TKI) with the exception of one patient with undetermined pathology who was treated with TKI alone (SC07). All treatments were administered prior to surgery in a neo-adjuvant setting and biopsies were collected after therapy. Detailed clinical data and treatment regimen for each patient is included in Table S1.

### Example 9—Whole Exome Sequencing

Libraries for whole exome sequencing were generated with TruSight Oncology DNA Library Prep Kit with 40 ng input DNA per sample. TruSight Oncology index PCR products were directly used for enrichment and target exome enrichment was performed using the IDT xGen Universal Blockers and IDT xGen Exome Research panel. A single-plex hybridization was done overnight at 65° C. Accuclear dsDNA Ultra High Sensitivity assay (Biotium) was used for library quantification of the Post-enriched libraries. Post enrichment libraries were normalized using bead-based normalization and pooled. Samples were sequenced with 101 bp paired-end reads on illumina NovaSeq™ 6000 S4 flow cell using the XP workflow for individual lane loading (12-plex per lane). On average, each sample yielded 500 million reads and MEDIAN_TARGET_COVERAGE depth of 360X.

### Example 10—Whole Transcriptome Sequencing

Libraries for whole transcriptome RNA-seq were generated with Illumina TruSeq Stranded Total RNA. 100 ng RNA was used as input for Ribo-Zero rRNA Removal Kit, with Illumina TruSeq RNA UD Indexes (96 indexes) for sample indexing. Qubit dsDNA High Sensitivity assay (Thermo Fisher Scientific) was used for library quantification. Sequencing was done on Illumina NovaSeq™ 6000 S2 (36-plex) or S4 (72-plex) flow cell with 76 bp paired-end sequencing to produce ˜200 million paired reads per library.

### Example 11—T-Cell Repertoire Sequencing

Libraries for T-cell repertoire sequencing were generated with AmpliSeq for Illumina Library PLUS paired with AmpliSeq cDNA Synthesis for Illumina with 100 ng RNA input per cDNA synthesis reaction. The TCR beta-SR Panel was used for generating amplicons, and AmpliSeq CD Indexes Set A for Illumina were used for sample barcodes. Qubit dsDNA High Sensitivity assay (Thermo Fisher Scientific) was used for library quantification. Sequencing was done on the NextSeq 550 (41-plex) with 151 bp paired-end sequencing to produce ˜5 million paired reads per library.

### Example 12—Metabolomics Sample Preparation

Samples were thawed and extracted according to Metabolon's standard protocol, which removed proteins, dislodged small molecules bound to the protein or physically trapped in the protein matrix, and recovered a wide range of chemically diverse metabolites. Samples were then frozen, dried under vacuum and prepared for LC/MS.

### Example 13—LC/MS

The Waters ACQUITY UPLC and the Thermo-Finnigan LTQ mass spectrometer, which consisted of an electrospray ionization (ESI) source and linear ion-trap (LIT) mass analyzer, were used for the LC/MS portion of the Metabolon platform. The sample extract was split in two and reconstituted in acidic and basic LC-compatible solvents. The acidic extracts were gradient eluted using water and methanol containing 0.1% Formic acid, while the basic extracts, which also used water and methanol, contained 6.5 mM Ammonium Bicarbonate. One aliquot was analyzed using acidic positive ion optimized conditions and the other used basic negative ion optimized conditions. The aliquots were two independent injections using separate dedicated columns. The MS analysis alternated between MS and data-dependent MS/MS scans using dynamic exclusion.

### Example 14—Mass Determination and MS/MS Fragmentation

The LC/MS portion of the platform was based on a Waters ACQUITY UPLC and a Thermo-Finnigan LTQ-FT mass spectrometer, which had a linear ion-trap (LIT) front end and a Fourier transform ion cyclotron resonance (FT-ICR) mass spectrometer backend. Accurate mass measurements could be performed for ions with counts greater than 2 million. The average mass error was less than 5 ppm. Ions with less than 2 million counts required a greater effort to characterize. Typically, fragmentation spectra (MS/MS) were generated in a data-dependent manner, but targeted MS/MS could be employed if necessary, such as in the case of lower-level signals.

### Example 15—Data Extraction and Quality Assurance

Data was extracted from the raw mass spectrometry files, which was loaded into a relational database. The information was then examined, and appropriate QC limits were imposed. Metabolon's proprietary peak integration software was used to identify peaks, and component parts were stored in a separate data structure.

### Example 16—Compound Identification

Metabolites were compared to an in-house library of standards from Metabolon. Data on each of these standards were based on retention index, mass-to-charge ratio, and MS/MS spectra. These parameters of each feature for each compound in the metabolomic data were compared to analogous parameters in the library. As described in Evans et al. (2009), compounds were identified based on three criteria: retention index within 75 RI units of the proposed identification, mass within 0.4 m/s and MS/MS forward and reverse match scores.

### Example 17—Data Normalization and Imputation

Each compound was corrected in run-day blocks by registering the medians to equal one and normalizing each data point accordingly. The data was subsequently log 2 normalized. When metabolite levels were below the level of detection, the lowest measured abundance of that compound across all samples was imputed.

### Example 18—RNA-Seq Pipeline

RNA-seq raw read sequences were aligned against human genome assembly hg19 by STAR 2-pass alignment (Dobin et al., 2013). QC metrics, for example general sequencing statistics, gene feature and body coverage, were then calculated based on the alignment result through RSeQC. RNA-seq gene level count values were computed by using the R package GenomicAlignments (Lawrence et al., 2013) over aligned reads with UCSC KnownGene (Karolchik et al., 2003) in hg19 as the base gene model. The union counting mode was used and only mapped paired reads after alignment quality filtering were considered. Finally, gene level FPKM (Fragments Per Kilobase Million) and raw read count values were computed by the R package DESeq2 (Love et al., 2014).

### Example 19—ESTIMATE

The ESTIMATEScore, which is the estimate of the presence of stromal and immune cells in tumor tissue, is calculated through the ESTIMATE R package (Yoshihara et al., 2013) based on a given gene expression profile in FPKM.

### Example 20—Immune Deconvolution Analysis

Two distinct popular computational methods, ssGSEA (Barbie et al., 2009) and CIBERSORT (Newman et al., 2015), were chosen for immune deconvolution analysis. Signature gene lists of immune cell types for ssGSEA were obtained from Bindea et al. (Bindea et al., 2013) and Senbabaoglu et al. (Senbabaoglu et al., 2016). ssGSEA takes the sample FPKM RNA-seq expression values as the input and computes an enrichment score for the given gene list of immune cell type relative to all other genes in the transcriptome. On the other hand, CIBERSORT also takes FPKM RNA-seq expression values as the input but uses a signature gene expression matrix of interest immune cell types instead to compute the infiltration level of each immune cell type. The LM22 immune cell signature which was validated and published along with CIBERSORT is used.

### Example 21—HERV Quantification

We used WTS to quantify HERVs as described before (Golkaram et al., 2021). Briefly, all RNAseq reads were aligned (using STAR aligner with optimized multi mapping options) to a custom genome built were human reference (hg19) and HERV specific reference are combined. Then reads aligned to non-HERV genes are removed and the rest are annotated.

### Example 22—WES Analysis Pipeline

Raw sequencing data were aligned to the hg19 genome build using the Burrows-Wheeler Aligner (BWA) version 0.7.17 (Li and Durbin, 2009). Further indel realignment, base-quality score recalibration and duplicate-read removal were performed using the Genome Analysis Toolkit (GATK) version 3.8 (McKenna et al., 2010) following raw reads alignments guidelines (DePristo et al., 2011). VarScan 2 (Koboldt et al., 2012), Strelka v2.9.10 (Kim et al., 2018), Platypus 0.8.1 (Rimmer et al., 2014), Mutect2—part of GATK 4.1.4.1 (DePristo et al., 2011), Somatic Sniper version 1.0.5.0 (SNVs only), and (Larson et al., 2012) were used for small variant calling and combination of 2 out 5 callers are reported as per Cancer Genome Atlas Research Network recommendations (Ellrott et al., 2018). Variants were filtered using the following criteria:
1) Tcov>10 & Taf>=0.04 & Ncov>7 & Naf<=0.01 & Tac>4 are set to Pass
2) Common SNPs are eliminated by comparison to snp142.vcf
3) Rare variants found in dbSNP are kept if Naf=0
4) Variants with Tcov<20 or Tac<4 are marked as low_confidence
5) Only variants called by more than 1 caller are reported.
6) Common variables gnomAD v 2.1.1 are excluded.

Variants were annotated using Ensembl Variant Effect Predictor (VEP) (McLaren et al., 2016). Additional optimization and filtering are applied for INDELS. INDELS in blacklisted regions (https://www.encodeproject.org/annotations/ENCSR636HFF/) and low mappability regions (such as repeat maskers) are excluded as per (Amemiya et al., 2019). Combination of filtered SNV and INDELS are used by maftools R package is used to generate oncoplots and summary plots, as per author's recommendations https://www.bioconductor.org/packages/release/bioc/vignettes/maftools/inst/doclmaftools.ht ml.

All nonsynonymous point mutations identified as above were translated into strings of 17 amino acids with the mutant amino acid situated centrally using a bioinformatics tool called NAseek. A sliding window method is used to identify the 8-11 amino acid substrings within the mutant 17-mer that had a predicted MHC Class I binding affinity of ≤2% Rank to one (or more) of the patient-specific HLA alleles. Binding affinity for the mutant and corresponding wild type nonamer is analyzed using NetMHCpan4.0 software. Only neoantigens with a TPM>1 are considered to be expressed.

Allele-specific copy number analysis is done by the FACETS v.6.1 (Shen and Seshan, 2016). Allele specific HLA loss is determined using LOHHLA as described before (McGranahan et al., 2017).

### Example 23—Intratumor Metabolic and RNA Heterogeneity Scores

Metabolite/gene- and patient-wise intra-patient heterogeneity scores were calculated using multi-region data. Data was first median-centered to remove any metabolite-level bias. For each metabolite, the difference between each pair of samples from the same tumor were calculated. The median difference between the paired-differences was then taken, yielding a metabolite/gene-specific, patient-specific measure of heterogeneity. This was repeated for all metabolites/genes, across all tumors, generating a matrix of metabolite/gene by patient values. Metabolite/gene ITH values are summarized as the median value per metabolite/gene across all tumors in the cohort. Patient ITH values are summarized as the median value per tumor across all metabolites. Patient ITH values represent the expected value of the absolute log-fold change for a randomly chosen metabolite within a given tumor.

### Example 24—ccRCC Evolutionary Subtypes and Intratumor DNA Heterogeneity Score

DNA ITH score is calculated as the ratio of subclonal to clonal driver genomic alterations including SNVs, INDELs, and SCNA (Turajlic et al., 2018b). A genomic alteration is defined to be subclonal if it is present in less than half of the regions collected in each patient. Patients who enough DNA biopsies are collected are classified into 1 of the 7 ccRCC evolutionary subtypes as described before (Turajlic et al., 2018b).

### Example 25—HLA and TCR Diversity

Shannon entropy is calculated to define TCR diversity (Wu et al., 2020). We used MiXCR on Illumna BaseSpace (http://basespace.illumina.com/apps/) for alignment and T cell clonotype identification. Immunarch (https://immunarch.com/) (Nazarov, 2020) was used for downstream analysis including visualization and data analysis. Morisita index (Horn, 1966) was used to measure clonotype overlap. HLA diversity index is measured as adopted from (Chowell et al., 2019) as described in (Golkaram et al., 2021).

### Example 26—Neoantigen Depletion

The fraction of neoantigens edited is defined for each sample where pretreatment data was available. We first calculated the neoantigen depletion as the number of neoantigens that were undetectable after therapy but were detected pretreatment. The fraction of neoantigens edited was then defined as the ratio of the total number of depleted neoantigens over total pretreatment neoantigens. To distinguish neoantigen depletion due to contraction from evasion, we exclude any neoantigens that were depleted without the presence of HLA LOH (defects in antigen presentation machinery), or reduced expression i.e. log 2(FC)<−1 where FC is the fold change defined as the ratio of post treatment TPM over pretreatment TPM after correction for tumor purity.

Likewise, HERV editing is defined as the median change in the expression of immunogenic HERVs compared to pre-treatment expression. Immunogenic HERVs refers to HERV loci whose expression strongly correlates with TIL abundance, FDR<0.05.

### Example 27—Weighted Gene Co-Expression Network Analysis (WGCNA) and Gene Signature Extraction

We performed WGCNA (Langfelder and Horvath, 2008) on all samples where the fraction of neoantigens edited was available similar to previously described (Motzer et al., 2020b). Briefly, genes with low expression values and invariant genes, that is, genes that were expressed in <5% of samples or had s.d.≤1 for expression (log 2 TPM) were filtered together with non-coding genes. The soft power of 6 was chosen based on goodness of fit to a scale-free network. We first annotate modules as JAVELIN or angiogenesis according to the Spearman correlation between the module eigengene and JAVELIN or angiogenesis ssGSEA scores (highest correlation is classified as JAVELIN or angiogenesis module). Likewise, among all modules, the module with the highest Spearman correlation with the fraction of neoantigens edited was annotated as immune escape module. Several genes (ADAMTS14, MMP11, FN1, COL5A1, COL5A2 and TIMP1) in this signature has previously been described as TGF-β-associated extracellular matrix genes that are linked to immune evasion and immunotherapy failure (Chakravarthy et al., 2018).

### Example 28—Statistical Analysis

All statistical tests were performed in R. To calculate correlations, cor.test with Spearman's method was used. Tests comparing distributions were performed using wilcox.test. All statistical analyses were two-sided and p-values were Benjamini-Hochberg corrected.

### REFERENCES

1. Amemiya, H. M., Kundaje, A., and Boyle, A. P. (2019). The ENCODE blacklist, identification of problematic regions of the genome. Scientific reports 9, 1-5.
2. Barbie, D. A., Tamayo, P., Boehm, J. S., Kim, S. Y., Moody, S. E., Dunn, I. F., Schinzel, A. C., Sandy, P., Meylan, E., and Scholl, C. (2009). Systematic RNA interference reveals that oncogenic KRAS-driven cancers require TBK1. Nature 462, 108-112.
3. Bindea, G., Mlecnik, B., Tosolini, M., Kirilovsky, A., Waldner, M., Obenauf, A. C., Angell, H., Fredriksen, T., Lafontaine, L., and Berger, A. (2013). Spatiotemporal dynamics of intratumoral immune cells reveal the immune landscape in human cancer. Immunity 39, 782-795.
4. Braun, D. A., Hou, Y., Bakouny, Z., Ficial, M., Sant'Angelo, M., Forman, J., Ross-Macdonald, P., Berger, A. C., Jegede, O. A., and Elagina, L. (2020). Interplay of somatic alterations and immune infiltration modulates response to PD-1 blockade in advanced clear cell renal cell carcinoma. Nature medicine 26, 909-918.
5. Chakravarthy, A., Khan, L., Bensler, N. P., Bose, P., and De Carvalho, D. D. (2018). TGF-β-associated extracellular matrix genes link cancer-associated fibroblasts to immune evasion and immunotherapy failure. Nature communications 9, 1-10.
6. Cherkasova, E., Malinzak, E., Rao, S., Takahashi, Y., Senchenko, V. N., Kudryavtseva, A. V., Nickerson, M. L., Merino, M., Hong, J. A., and Schrump, D. S. (2011). Inactivation of the von Hippel-Lindau tumor suppressor leads to selective expression of a human endogenous retrovirus in kidney cancer. Oncogene 30, 4697-4706.
7. Chiappinelli, K. B., Strissel, P. L., Desrichard, A., Li, H., Henke, C., Akman, B., Hein, A., Rote, N. S., Cope, L. M., and Snyder, A. (2015). Inhibiting DNA methylation causes an interferon response in cancer via dsRNA including endogenous retroviruses. Cell 162, 974-986.
8. Choueiri, T. K., and Kaelin, W. G. (2020). Targeting the HIF2-VEGF axis in renal cell carcinoma. Nature Medicine 26, 1519-1530.
9. Chowell, D., Krishna, C., Pierini, F., Makarov, V., Rizvi, N. A., Kuo, F., Morris, L. G., Riaz, N., Lenz, T. L., and Chan, T. A. (2019). Evolutionary divergence of HLA class I genotype impacts efficacy of cancer immunotherapy. Nature medicine 25, 1715-1720.
10. Chowell, D., Morris, L. G., Grigg, C. M., Weber, J. K., Samstein, R. M., Makarov, V., Kuo, F., Kendall, S. M., Requena, D., and Riaz, N. (2018). Patient HLA class I genotype influences cancer response to checkpoint blockade immunotherapy. Science 359, 582-587.
11. Clark, D. J., Dhanasekaran, S. M., Petralia, F., Pan, J., Song, X., Hu, Y., da Veiga Leprevost, F., Reva, B., Lih, T.-S. M., and Chang, H.-Y. (2019). Integrated proteogenomic characterization of clear cell renal cell carcinoma. Cell 179, 964-983. e931.
12. DePristo, M. A., Banks, E., Poplin, R., Garimella, K. V., Maguire, J. R., Hartl, C., Philippakis, A. A., Del Angel, G., Rivas, M. A., and Hanna, M. (2011). A framework for variation discovery and genotyping using next-generation DNA sequencing data. Nature genetics 43, 491.
13. Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
14. Ellrott, K., Bailey, M. H., Saksena, G., Covington, K. R., Kandoth, C., Stewart, C., Hess, J., Ma, S., Chiotti, K. E., and McLellan, M. (2018). Scalable open science approach for mutation calling of tumor exomes using multiple genomic pipelines. Cell systems 6, 271-281. e277.
15. Gerlinger, M., Horswell, S., Larkin, J., Rowan, A. J., Salm, M. P., Varela, I., Fisher, R., McGranahan, N., Matthews, N., and Santos, C. R. (2014). Genomic architecture and evolution of clear cell renal cell carcinomas defined by multiregion sequencing. Nature genetics 46, 225.
16. Ghatalia, P., and Rathmell, W. K. (2018). Systematic review: clearcode 34-a validated prognostic signature in clear cell renal cell carcinoma (ccRCC). Kidney cancer 2, 23-29.
17. Golkaram, M., Salmans, M. L., Kaplan, S., Vijayaraghavan, R., Martins, M., Khan, N., Garbutt, C., Wise, A., Yao, J., and Casimiro, S. (2021). HERVs establish a distinct molecular subtype in stage II/III colorectal cancer with poor outcome. NPJ genomic medicine 6,1-11.
18. Haake, S. M., Brooks, S. A., Welsh, E., Fulp, W. J., Chen, D.-T., Dhillon, J., Haura, E., Sexton, W., Spiess, P. E., and Pow-Sang, J. (2016). Patients with ClearCode34-identified molecular subtypes of clear cell renal cell carcinoma represent unique populations with distinct comorbidities. Paper presented at: Urologic Oncology: Seminars and Original Investigations (Elsevier).
19. Hakimi, A. A., Attalla, K., DiNatale, R G., Ostrovnaya, I., Flynn, J., Blum, K. A., Ged, Y., Hoen, D., Kendall, S. M., and Reznik, E. (2020). A pan-cancer analysis of PBAF complex mutations and their association with immunotherapy response. Nature communications 11, 1-11.
20. Havel, J. J., Chowell, D., and Chan, T. A. (2019). The evolving landscape of biomarkers for checkpoint inhibitor immunotherapy. Nature Reviews Cancer 19, 133-150.
21. Hegde, P. S., and Chen, D. S. (2020). Top 10 challenges in cancer immunotherapy. Immunity 52, 17-35.
22. Horn, H. S. (1966). Measurement of“overlap” in comparative ecological studies. The American Naturalist 100, 419-424.
23. Huang, Y., Wang, J., Jia, P., Li, X., Pei, G., Wang, C., Fang, X., Zhao, Z., Cai, Z., and Yi, X. (2019). Clonal architectures predict clinical outcome in clear cell renal cell carcinoma. Nature communications 10, 1-10.
24. Jonasch, E., Walker, C. L., and Rathmell, W. K. (2020). Clear cell renal cell carcinoma ontogeny and mechanisms of lethality. Nature Reviews Nephrology, 1-17.
25. Karolchik, D., Baertsch, R., Diekhans, M., Furey, T. S., Hinrichs, A., Lu, Y., Roskin, K. M., Schwartz, M., Sugnet, C. W., and Thomas, D. J. (2003). The UCSC genome browser database. Nucleic acids research 31, 51-54.
26. Kim, S., Scheffler, K., Halpern, A. L., Bekritsky, M. A., Noh, E., Killberg, M., Chen, X., Kim, Y., Beyter, D., and Krusche, P. (2018). Strelka2: fast and accurate calling of germline and somatic variants. Nature methods 15, 591-594.
27. Koboldt, D. C., Zhang, Q., Larson, D. E., Shen, D., McLellan, M. D., Lin, L., Miller, C. A., Mardis, E. R., Ding, L., and Wilson, R. K. (2012). VarScan 2: somatic mutation and copy number alteration discovery in cancer by exome sequencing. Genome research 22, 568-576.
28. Krishna, C., DiNatale, R. G., Kuo, F., Srivastava, R. M., Vuong, L., Chowell, D., Gupta, S., Vanderbilt, C., Purohit, T. A., and Liu, M. (2021). Single-cell sequencing links multiregional immune landscapes and tissue-resident T cells in ccRCC to tumor topology and therapy efficacy. Cancer Cell.
29. Langfelder, P., and Horvath, S. (2008). WGCNA: an R package for weighted correlation network analysis. BMC bioinformatics 9, 1-13.
30. Larson, D. E., Harris, C. C., Chen, K., Koboldt, D. C., Abbott, T. E., Dooling, D. J., Ley, T. J., Mardis, E. R., Wilson, R. K., and Ding, L. (2012). SomaticSniper: identification of somatic point mutations in whole genome sequencing data. Bioinformatics 28, 311-317.
31. Lawrence, M., Huber, W., Pages, H., Aboyoun, P., Carlson, M., Gentleman, R., Morgan, M. T., and Carey, V. J. (2013). Software for computing and annotating genomic ranges. PLoS Comput Biol 9, e1003118.
32. Levine, J. H., Simonds, E. F., Bendall, S. C., Davis, K. L., El-ad, D. A., Tadmor, M. D., Litvin, O., Fienberg, H. G., Jager, A., and Zunder, E. R. (2015). Data-driven phenotypic dissection of AML reveals progenitor-like cells that correlate with prognosis. Cell 162, 184-197.
33. Li, H., and Durbin, R. (2009). Fast and accurate short read alignment with Burrows-Wheeler transform. bioinformatics 25, 1754-1760.
34. Liu, X.-D., Hoang, A., Zhou, L., Kalra, S., Yetil, A., Sun, M., Ding, Z., Zhang, X., Bai, S., and German, P. (2015). Resistance to antiangiogenic therapy is associated with an immunosuppressive tumor microenvironment in metastatic renal cell carcinoma. Cancer immunology research 3, 1017-1029.
35. Liu, X.-D., Kong, W., Peterson, C. B., McGrail, D. J., Hoang, A., Zhang, X., Lam, T., Pilie, P. G., Zhu, H., and Beckermann, K. E. (2020). PBRM1 loss defines a nonimmunogenic tumor phenotype associated with checkpoint inhibitor resistance in renal carcinoma. Nature communications 11, 1-14.
36. Love, M. I., Huber, W., and Anders, S. (2014). Moderated estimation of fold change and dispersion for RNA-seq data with DESeq2. Genome biology 15, 1-21.
37. McDermott, D. F., Huseni, M. A., Atkins, M. B., Motzer, R. J., Rini, B. I., Escudier, B., Fong, L., Joseph, R. W., Pal, S. K., and Reeves, J. A. (2018). Clinical activity and molecular correlates of response to atezolizumab alone or in combination with bevacizumab versus sunitinib in renal cell carcinoma. Nature medicine 24, 749-757.
38. McGranahan, N., Rosenthal, R., Hiley, C. T., Rowan, A. J., Watkins, T. B., Wilson, G. A., Birkbak, N. J., Veeriah, S., Van Loo, P., and Herrero, J. (2017). Allele-specific HLA loss and immune escape in lung cancer evolution. Cell 171, 1259-1271. e1211.
39. McKenna, A., Hanna, M., Banks, E., Sivachenko, A., Cibulskis, K., Kernytsky, A., Garimella, K., Altshuler, D., Gabriel, S., and Daly, M. (2010). The Genome Analysis Toolkit: a MapReduce framework for analyzing next-generation DNA sequencing data. Genome research 20, 1297-1303.
40. McLaren, W., Gil, L., Hunt, S. E., Riat, H. S., Ritchie, G. R, Thormann, A., Flicek, P., and Cunningham, F. (2016). The ensembl variant effect predictor. Genome biology 17, 1-14.
41. Montesion, M., Murugesan, K., Jin, D. X., Sharaf, R., Sanchez, N., Guria, A., Minker, M., Li, G., Fisher, V., and Sokol, E. S. (2021). Somatic HLA class I loss is a widespread mechanism of immune evasion which refines the use of tumor mutational burden as a biomarker of checkpoint inhibitor response. Cancer discovery 11, 282-292.
42. Motzer, R. J., Banchereau, R, Hamidi, H., Powles, T., McDermott, D., Atkins, M. B., Escudier, B., Liu, L.-F., Leng, N., and Abbas, A. R. (2020a). Molecular subsets in renal cancer determine outcome to checkpoint and angiogenesis blockade. Cancer Cell 38, 803-817. e804.
43. Motzer, R. J., Robbins, P. B., Powles, T., Albiges, L., Haanen, J. B., Larkin, J., Mu, X. J., Ching, K. A., Uemura, M., and Pal, S. K. (2020b). Avelumab plus axitinib versus sunitinib in advanced renal cell carcinoma: Biomarker analysis of the phase 3 JAVELIN Renal 101 trial. Nature medicine 26, 1733-1741.
44. Motzer, R J., Tannir, N. M., McDermott, D. F., Frontera, O. A., Melichar, B., Choueiri, T. K., Plimack, E. R., Barthélémy, P., Porta, C., and George, S. (2018). Nivolumab plus ipilimumab versus sunitinib in advanced renal-cell carcinoma. New England Journal of Medicine.
45. Nazarov, V. (2020). immunarch. bot & Eugene Rumynskiy. immunomind/immunarch: 0.6. 5: Basic single-cell support. In, (Zenodo).
46. Nelson, P. N., Carnegie, P., Martin, J., Ejtehadi, H. D., Hooley, P., Roden, D., Rowland-Jones, S., Warren, P., Astley, J., and Murray, P. G. (2003). Demystified . . . Human endogenous retroviruses. Molecular Pathology 56, 11.
47. Newman, A. M., Liu, C. L., Green, M. R., Gentles, A. J., Feng, W., Xu, Y., Hoang, C. D., Diehn, M., and Alizadeh, A. A. (2015). Robust enumeration of cell subsets from tissue expression profiles. Nature methods 12, 453-457.
48. Otto, G. (2018). PBRM1 loss promotes tumour response to immunotherapy. Nature Reviews Clinical Oncology 15, 134-135.
49. Reuben, A., Gittelman, R., Gao, J., Zhang, J., Yusko, E. C., Wu, C.-J., Emerson, R., Zhang, J., Tipton, C., and Li, J. (2017). TCR repertoire intratumor heterogeneity in localized lung adenocarcinomas: an association with predicted neoantigen heterogeneity and postsurgical recurrence. Cancer discovery 7, 1088-1097.
50. Riley, T. P., Keller, G. L., Smith, A. R., Davancaze, L. M., Arbuiso, A. G., Devlin, J. R., and Baker, B. M. (2019). Structure based prediction of neoantigen immunogenicity. Frontiers in immunology 10, 2047.
51. Rimmer, A., Phan, H., Mathieson, I., Iqbal, Z., Twigg, S. R, Wilkie, A. O., McVean, G., and Lunter, G. (2014). Integrating mapping-, assembly- and haplotype-based approaches for calling variants in clinical sequencing applications. Nature genetics 46, 912-918.
52. Rosenthal, R., Cadieux, E. L., Salgado, R., A1 Bakir, M., Moore, D. A., Hiley, C. T., Lund, T., Tanic, M., Reading, J. L., and Joshi, K. (2019). Neoantigen-directed immune escape in lung cancer evolution. Nature 567, 479-485.
53. Senbabaoglu, Y., Gejman, R S., Winer, A. G., Liu, M., Van Allen, E. M., de Velasco, G., Miao, D., Ostrovnaya, I., Drill, E., and Luna, A. (2016). Tumor immune microenvironment characterization in clear cell renal cell carcinoma identifies prognostic and immunotherapeutically relevant messenger RNA signatures. Genome biology 17, 1-25.
54. Shen, R, and Seshan, V. E. (2016). FACETS: allele-specific copy number and clonal heterogeneity analysis tool for high-throughput DNA sequencing. Nucleic acids research 44, e131-e131.
55. Smith, C. C., Beckermann, K. E., Bortone, D. S., De Cubas, A. A., Bixby, L. M., Lee, S. J., Panda, A., Ganesan, S., Bhanot, G., and Wallen, E. M. (2019). Endogenous retroviral signatures predict immunotherapy response in clear cell renal cell carcinoma. The Journal of clinical investigation 128, 4804-4820.
56. Turajlic, S., and Swanton, C. (2017). TRACERx Renal: tracking renal cancer evolution through therapy. Nature Reviews Urology 14, 575-577.
57. Turajlic, S., Xu, H., Litchfield, K., Rowan, A., Chambers, T., Lopez, J. I., Nicol, D., O'Brien, T., Larkin, J., and Horswell, S. (2018a). Tracking cancer evolution reveals constrained routes to metastases: TRACERx renal. Cell 173, 581-594. e512.
58. Turajlic, S., Xu, H., Litchfield, K., Rowan, A., Horswell, S., Chambers, T., O'Brien, T., Lopez, J. I., Watkins, T. B., and Nicol, D. (2018b). Deterministic evolutionary trajectories influence primary tumor growth: TRACERx renal. Cell 173, 595-610. e511.
59. Van Allen, E. M., and Choueiri, T. K. (2020). Dissecting the immunogenomic biology of cancer for biomarker development. Nature Reviews Clinical Oncology, 1-2.
60. Vitale, I., Shema, E., Loi, S., and Galluzzi, L. (2021). Intratumoral heterogeneity in cancer progression and response to immunotherapy. Nature medicine, 1-13.
61. Williams, J. B., Li, S., Higgs, E. F., Cabanov, A., Wang, X., Huang, H., and Gajewski, T. F. (2020). Tumor heterogeneity and clonal cooperation influence the immune selection of IFN-γ-signaling mutant cancer cells. Nature communications 11, 1-14.
62. Wu, T. D., Madireddi, S., de Almeida, P. E., Banchereau, R, Chen, Y.-J. J., Chitre, A. S., Chiang, E. Y., Iftikhar, H., O'Gorman, W. E., and Au-Yeung, A. (2020). Peripheral T cell expansion predicts tumour infiltration and clinical response. Nature 579, 274-278.
63. Xu, C., and Su, Z. (2015). Identification of cell types from single-cell transcriptomes using a novel clustering method. Bioinformatics 31, 1974-1980.
64. Yarchoan, M., Hopkins, A., and Jaffee, E. M. (2017). Tumor mutational burden and response rate to PD-1 inhibition. The New England journal of medicine 377, 2500.
65. Yoshihara, K., Shahmoradgoli, M., Martinez, E., Vegesna, R., Kim, H., Torres-Garcia, W., Trevifto, V., Shen, H., Laird, P. W., and Levine, D. A. (2013). Inferring tumour purity and stromal and immune cell admixture from expression data. Nature communications 4,1-11.
66. Zhang, A. W., McPherson, A., Milne, K., Kroeger, D. R., Hamilton, P. T., Miranda, A., Funnell, T., Little, N., de Souza, C. P., and Laan, S. (2018). Interfaces of malignant and immunologic clonal dynamics in ovarian cancer. Cell 173, 1755-1769. el722.
67. Zhang, J., Fujimoto, J., Yusko, E., Zhang, J., Vignali, M., Song, X., Rodriguez-Canales, J., Parra Cuentas, E. R., Behrens, C., and Benzeno, S. (2016). Intratumor heterogeneity of T cell receptor repertoire in lung cancers and its association with tumor genomic profile. In, (American Society of Clinical Oncology).

What is claimed is:

1. A method for determining an intra-tumoral heterogeneity (ITH) in a tumor microenvironment (TME) of a cancer, the method comprising:
providing a biopsy sample from a cancer patient;
ascertaining a weighted genome instability index (wGII) based on a mutational frequency in a plurality of regions within the biopsy sample;
comparing the wGII to a first median value and classifying the plurality of regions within the biopsy sample as having either higher wGII than the first median value or lower wGII than the first median value; and
analyzing a plurality of parameters in the plurality of regions within the biopsy sample, classified as having either higher wGII than the first median value or lower wGII than the first median value, to determine an ITH index of each of the plurality of parameters of each of the plurality of regions within the biopsy sample.

2. The method of claim 1, further comprising:
comparing the ITH index of each of the plurality of parameters to a second median value; and
classifying each of the plurality of parameters as having either a higher ITH index than the second median value or a lower ITH index than the second median value,
wherein a higher ITH index than the second median value correlates with a higher wGII in each of the plurality of regions within the biopsy sample, and
wherein a lower ITH index than the second median value correlates with a lower wGII in each of the plurality of regions within the biopsy sample.

3. The method of claim 1, wherein the cancer is clear cell renal cell carcinoma (ccRCC).

4. The method of claim 1, wherein the biopsy sample is a nephrectomy sample.

5. The method of claim 1, wherein analyzing the plurality of parameters in the plurality of regions within the biopsy sample is performed by collecting at least one of DNA, RNA, cellular fractions, tissue sections, and tissue extracts for each of the plurality of regions within the biopsy sample.

6. The method of claim 1, wherein the plurality of parameters comprises genomic analysis, transcriptomic analysis, TCR analysis, immune cell analysis, metabolomics analysis, pathology analysis, myeloid signature analysis, JAVELIN signature analysis, effector T cell signature analysis, antigen presentation signature analysis, and angiogenic signature analysis.

7. The method of claim 2, further comprising correlating the ITH index of each of the plurality of parameters with at least one ccRCC evolutionary subtype.

8. The method of claim 7, wherein the at least one ccRCC evolutionary subtype comprises a VHL wildtype, a VHL monodriver, multiple clonal driver, a BAP1 driver, or PBRM1 driven tumors.

9. The method of claim 8, wherein the PBRM1 driven tumors comprise PBRM1→SETD2, PBRM1→SCNA, and PBRM1→PI3K.

10. The method of claim 8, wherein the VHL monodriver and multiple clonal driver subtypes correlate with an ITH index that is lower than the second median value.

11. The method of claim 8, wherein PBRM1 driven tumors, SETD2 mutations, loss of heterozygosity (LOH) in Human Leukocyte Antigen (HLA), and loss of CDKN2A/B copy number correlates with an ITH index that is higher than the second median value.

12. The method of claim 8, wherein PBRM1 driven tumors are associated with elevated HERV expression.

13. The method of claim 1, further comprising ascertaining neoantigen heterogeneity by counting 8-11 amino acids length neoantigens in the plurality of regions within the biopsy sample.

14. The method of claim 13, wherein an ITH index that is higher than the second median value is associated with higher neoantigen editing in the plurality of regions within the biopsy sample.

15. The method of claim 13, wherein an ITH index that is lower than the second median value is associated with lower neoantigen editing in the plurality of regions within the biopsy sample.

16. The method of claim 1, further comprising administering ICB treatment to the at least one patient and ascertaining neoantigen heterogeneity in the plurality of regions within the biopsy sample prior to and after administering the ICB treatment.

17. The method of claim 16, wherein ascertaining neoantigen heterogeneity comprises counting 8-11 amino acids length neoantigens prior to and after administering the ICB treatment.

18. The method of claim 17, wherein a deletion of neoantigens after administering the ICB treatment is indicative of neoantigen editing by the ICB treatment.

19. The method of claim 17, wherein a deletion of neoantigens after administering the ICB treatment is indicative of selective pressure against hydrophobic residues and selection in favor of hydrophilic residues.

20. The method of claim 17, wherein a deletion of neoantigens after administering the ICB treatment is indicative of selective pressure against Phenylalanine and selection in favor of Arginine and Glutamic acid.

21. The method of claim 17, wherein a deletion of neoantigens after administering the ICB treatment correlates with an ITH index that is higher than the second median value.

22. The method of claim 17, wherein a deletion of neoantigens after administering the ICB treatment correlates with an immunosuppressive TME.

23. The method of any one of claims 16-22, wherein the ICB treatment is selected from the group consisting of Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

24. The method of claim 1, wherein an ITH index that is higher than the second median value correlates with high myeloid signature and low effector T cell signature.

25. The method of claim 1, wherein an ITH index that is higher than the second median value correlates with low antigen presentation signature.

26. The method of claim 1, wherein an ITH index that is higher than the second median value correlates with reduced TCR diversity.

27. The method of claim 1, wherein an ITH index that is lower than the second median value correlates with the biopsy being sparsely infiltrated by tumor infiltrating lymphocytes (TILs) and/or being infiltrated by stromal TILs.

28. The method of claim 1, wherein an ITH index that is higher than the second median value correlates with the biopsy being infiltrated by substantial levels of both epithelial and stromal TILs.

29. The method of claim 28, wherein infiltration of the biopsy by substantial levels of both epithelial and stromal TILs is correlated with an immune evasion/escape gene signature.

30. The method of claim 29, wherein immune evasion/escape is correlated with HLA LOH and CDKN2A/B loss in the cellular fractions collected from the plurality of regions within the biopsy sample.

31. The method of claim 29, wherein immune evasion/escape is correlated with HLA LOH and CDKN2A/B loss in a cellular fraction collected from peripheral blood of the patient.

32. The method of claim 6, wherein the genomic analysis comprises small variant calling, evaluation of somatic copy number alterations, allele specific copy number calling, HLA typing, and in silico binding prediction of putative neoantigens.

33. The method of claim 6, wherein the transcriptomic analysis comprises quantification of gene expression data, a gene expression microarray, RT-PCR, and RNA-Seq.

34. The method of claim 6, wherein the TCR analysis comprises T cell clonotyping, T cell diversity estimation using a diversity index such as Shannon Entropy index, Simpson's Diversity index, and Berger Parker index.

35. The method of claim 6, wherein the immune cell analysis comprises gene signature analysis, such as, for example, a tumor microenvironment gene signature analysis. In some embodiments, the gene signature analysis comprises use of a gene set enrichment analysis, for example a single sample Gene Set Enrichment Analysis (ssGSEA) method, as is known in the art and exemplified by Barbie D A, Tamayo P, et al. Systematic RNA interference reveals that oncogenic KRAS-driven cancers require TBK1.

36. The method of claim 6, wherein the metabolomics analysis comprises quantification of major metabolites in a tumor tissue sample using liquid chromatography-mass spectrometry (LC-MS) and tandem mass spectroscopy (MS/MS) for quantification of metabolites in the tumor tissue sample.

37. The method of claim 6, wherein the pathology analysis comprises tumor-stroma-immune grading using a pathology methods to classify tumors to N-TIL (tumors sparsely infiltrated by TILs), S-TIL (tumors dominated by stromal TILs), and ES-TIL (tumors with substantial levels of both epithelial and stromal TILs).

38. The method of claim 6, wherein the myeloid signature analysis comprises ssGSEA analysis to evaluate enrichment of one or more of the following genes: IL6, CXCL1, CXCL2, CXCL3, CXCL8, and PTGS2.

39. The method of claim 6, wherein the JAVELIN signature analysis comprises ssGSEA analysis to evaluate enrichment of one or more of the following genes: NRARP, NRXN3, CALCRL, TEK, ECSCR, PTPRB, CD34, RAMP2, KDR, NOTCH4, FLT1, GJA5, TBX2, HEY2, ARHGEF15, SMAD6, AQP1, GATA2, ENPP2, ATP1A2, EDNRB, VIP, KCNAB1, RAMP3, CACNB2, and CASQ2.

40. The method of claim 6, wherein the effector T cell signature analysis comprises ssGSEA analysis to evaluate enrichment of one or more of the following genes: CD8A, EOMES, PRF1, IFNG, and CD274.

41. The method of claim 6, wherein the antigen presentation signature analysis comprises ssGSEA analysis to evaluate enrichment of one or more of the following genes: HLA-A, HLA-B, HLA-C, B2M, TAP1, TAP2, and TAPBP.

42. The method of claim 6, wherein the angiogenic signature analysis comprises ssGSEA analysis to evaluate enrichment of one or more of the following genes: VEGFA, KDR, ESM1, PECAM1, ANGPTL4, and CD34.

43. The method of claim 29, wherein the immune evasion/escape gene signature comprises ssGSEA analysis to evaluate enrichment of one or more of the following genes: TIMP1, PXDN, COL15A1, OLFML2B, COL5A2, DLX5, SOX11, KLHDC8A, UNC5A, ADAMTS14, MMP11, and FN1.

44. A method of predicting an outcome of immune checkpoint inhibitor (ICB) therapy, the method comprising:
providing a biopsy sample from a cancer patient;
ascertaining a weighted genome instability index (wGII) based on a mutational frequency in a plurality of regions within the biopsy sample;
comparing the wGII to a first median value and classifying the plurality of regions within the biopsy sample as having either a higher wGII than the first median value or a lower wGII than first median value;
analyzing a plurality of parameters in the plurality of regions within the biopsy sample, classified as having either higher wGII than median or lower wGII than median, to determine an ITH index of each of the plurality of parameters of each of the plurality of regions within the biopsy sample;
comparing the ITH index of each of the plurality of parameters to a second median value; and
classifying each of the plurality of parameters as having either higher ITH index than the second median value or a lower ITH index than the second median value,
wherein a higher ITH index than the second median value is predictive of the patient being nonresponsive to ICB therapy, and
wherein a lower ITH index than the second median value is predictive of the patient being responsive to ICB therapy.

45. The method of claim 44, wherein the cancer is clear cell renal cell carcinoma (ccRCC).

46. The method of claim 44, wherein the biopsy sample is a nephrectomy sample.

47. The method of claim 44, wherein analyzing the plurality of parameters in the plurality of regions within the biopsy sample is performed by at least one of collecting DNA, RNA, cellular fraction, tissue section, and tissue extraction for each of the plurality of regions within the biopsy sample.

48. The method of claim 44, wherein the ICB treatment is selected from the group consisting of: Pembrolizumab (Keytruda), Nivolumab (Opdivo), Cemiplimab (Libtayo) Atezolizumab (Tecentriq), Avelumab (Bavencio), Durvalumab (Imfinzi), and Ipilimumab (Yervoy).

49. The method of any of claims 1-48, further comprising administering ICB treatment to the cancer patient if the ITH index is lower than the second median value.

50. The method of any of claims 1-48, further comprising administering a non-ICB cancer therapy and not administering an ICB treatment to the cancer patient if the ITH index is higher than the second median value.
